(19) United States
(12) Patent Application Publication
(10) Pub. No.: US 2022/0333212 A1
(43) Pub. Date: Oct. 20, 2022

(54) METHODS OF IDENTIFYING SOMATIC MUTATIONAL SIGNATURES FOR EARLY CANCER DETECTION

(71) Applicant: GRAIL, Inc., Menlo Park, CA (US)

(72) Inventor: Oliver Claude Venn, III, San Francisco, CA (US)

(21) Appl. No.: 17/845,930

(22) Filed: Jun. 21, 2022

Related U.S. Application Data
(62) Division of application No. 15/806,310, filed on Nov. 7, 2017.
(60) Provisional application No. 62/569,519, filed on Oct. 7, 2017.
(60) Provisional application No. 62/469,984, filed on Mar. 10, 2017.
(60) Provisional application No. 62/418,639, filed on Nov. 7, 2016.

(51) Int. Cl. C12Q 1/6886; G06N 20/00; G16H 50/20; G16B 30/00; G16B 20/20; G16B 5/00; G16B 40/20; G16B 30/10; G16B 30/20; G16B 40/00

(52) U.S. Cl. CPC C12Q 1/6886; G06N 20/00; G16H 50/20; G16B 30/00; G16B 20/20; G16B 5/00; G16B 40/20; C12Q 2600/156; G16B 30/10; G16B 30/20; G16B 40/00

(57) ABSTRACT

Aspects of the invention include methods and systems for identifying somatic mutational signatures for detecting, diagnosing, monitoring and/or classifying cancer in a patient known to have, or suspected of having cancer. In various embodiments, the methods of the invention use a non-negative matrix factorization (NMF) approach to construct a signature matrix that can be used to identify latent signatures in a patient sample for detection and classification of cancer. In some embodiments, the methods of the invention may use principal components analysis (PCA) or vector quantization (VQ) approaches to construct a signature matrix.

### CROSS REFERENCE TO RELATED APPLICATIONS

This application is a divisional of U.S. application Ser. No. 15/806,310, filed Nov. 7, 2017, which claims priority benefit of the filing date of U.S. Provisional Patent Application No. 62/418,639, filed on Nov. 7, 2016, the disclosure of which application is herein incorporated by reference in its entirety. This application also claims priority benefit of the filing date of U.S. Provisional Patent Application No. 62/469,984, filed on Mar. 10, 2017, the disclosure of which application is herein incorporated by reference in its entirety. This application also claims priority benefit of the filing date of U.S. Provisional Patent Application No. 62/569,519, filed on Oct. 7, 2017, the disclosure of which application is herein incorporated by reference in its entirety.

### BACKGROUND OF THE INVENTION

Molecular analysis of circulating cell-free nucleic acids (e.g., cell-free DNA (cfDNA), cell-free RNA (cfRNA)) is increasingly recognized as a valuable approach to aid in detecting, diagnosing, monitoring and classifying cancer. In the last few years, DNA sequence analysis of cancer genomes has revealed distinct mutational signatures, representing a diversity of mutational processes underlying the development of cancer. Identification of underlying mutational signatures in a subject's cfDNA sample may provide valuable diagnostic information for cancer patients as well as provide a platform for early detection of cancer. There is a need for new methods for profiling a cfDNA sample for detecting, diagnosing, monitoring, and/or classifying cancer.

### SUMMARY OF THE INVENTION

Aspects of the invention include methods and systems for identifying somatic mutational signatures for detecting, diagnosing, monitoring and/or classifying cancer in a patient known to have, or suspected of having cancer. In various embodiments, the methods of the invention use a non-negative matrix factorization (NMF) approach to construct a signature matrix that can be used to identify latent signatures in a patient sample for detection and classification of cancer. In other embodiments, the methods of the invention may use principal components analysis (PCA) or vector quantization (VQ) approaches to construct a signature matrix. In one example, the patient sample is a cell-free nucleic acid sample (e.g., cell-free DNA (cfDNA) and/or cell-free RNA (cfRNA)).

The construction of a signature matrix using non-negative matrix factorization can be generalized to multiple features relevant to cancer detection and/or classification. In some embodiments, a signature matrix comprises a plurality of signatures where the probability of the occurrence for each of a plurality of features are represented. Examples of relevant features include, but are not limited to, an upstream sequence context of a base substitution mutation, a downstream sequence context of a base substitution mutation, an insertion, a deletion, a somatic copy number alteration (SCNA), a translocation, a genomic methylation status, a chromatin state, a sequencing depth of coverage, an early versus late replicating region, a sense versus antisense strand, an inter mutation distance, a variant allele frequency, a fragment start/stop, a fragment length, and a gene expression status, or any combination thereof. In one embodiment, the upstream and/or downstream sequence context can comprise a region of a nucleic acid that ranges in length from about 2 to about 40 bp, such as from about 3 to about 30 bp, such as from about 3 to about 20 bp, or such as from about 2 to about 10 bp of sequence context of a base substitution mutation. In one embodiment, the upstream and/or downstream sequence context may be a triplet sequence context, a quadruplet sequence context, a quintuplet sequence context, a sextuplet sequence context, or a septuplet sequence context of base substitution mutations. In some embodiments, the upstream and/or downstream sequence context can be the triplet sequence context of a base substitution mutation.

In one embodiment, the methods of the invention are used to identify latent somatic mutational signatures in a subject's (e.g., an asymptomatic subject) cfDNA sample for early detection of cancer.

In another embodiment, the methods of the invention are used to infer tissue of origin for a patient's cancer based on latent mutational signatures identified in the patient's cfDNA sample.

In yet another embodiment, the methods of the invention are used to identify latent mutational signatures in a patient's cfDNA sample that can be used to classify the patient for different types of therapies.

In yet another embodiment, non-negative matrix factorization is applied to learn error modes in a somatic variant (mutation) calling assay. For example, systematic errors (e.g., errors contributed during library preparation, PCR, hybridization capture, and/or sequencing) that underlie the assay can be identified and assigned unique signatures that can be used to distinguish between the contribution from true somatic variants and artifactual variants arising from the technical processes in the assay.

In yet another embodiment, non-negative matrix factorization can be used to identify mutational signatures that are associated with healthy aging. Mutation processes that are associated with aging are assigned mutational signatures that can be used to distinguish between healthy somatic mutations associated with patient age and somatic mutations contributed from, and indicative of, a cancer process in the patient.

In another embodiment, one or more mutational signatures can be monitored over time and used for diagnosing, monitoring, and/or classifying cancer. For example, the observed mutational profile in cfDNA from patient samples at two or more time points can be evaluated. In some embodiments, two or more mutational signature processes can be evaluated as a combination of different mutational signatures. In still another embodiment, one or more mutational signatures can be monitored over time (e.g., at a plurality of time points) to monitor the effectiveness of a therapeutic regimen or other cancer treatment.

Somatic mutations (i.e., driver mutations and passenger mutations) in a cancer genome are typically the cumulative consequence of one or more mutational processes of DNA damage and repair. Although not wishing to be bound by theory, it is believed that the strength and duration of exposure to each mutational process (e.g., environmental factors and DNA repair processes) results in a unique profile of somatic mutations in a subject (e.g., a cancer patient). These unique combinations of mutation types form a unique “mutational signature” for the cancer patient. Furthermore, as is well known in the art, a somatic mutation, or mutational profile can depend on the particular sequence context of the mutation. For example, UV damage typically results in a base change of C to T, when the base change occurs within a sequence context of (-T|C|-)C(A|T|C|G). In this example, C is the mutated base and the bases upstream (T or C) and downstream (A, T, C, or G) of C affect the probability of a mutation under UV radiation. In another example, spontaneous deamination of 5-methylcytosine typically results in a base change of C to T, when the base change occurs within a sequence context of (A|T|C|G)C(-|-|-|G). Accordingly, in one embodiment, the sequence context of identified mutations can be utilized as a feature for analyzing somatic mutations in the detection and/or classification of cancer.

### BRIEF DESCRIPTION OF THE DRAWINGS

FIG. 1 illustrates a flow diagram of a method for identifying somatic mutational signatures for detection of cancer, in accordance with the present invention;

FIG. 2 is a bar graph showing an example of a mutational profile from a patient's cfDNA sample;

FIG. 3 illustrates a schematic diagram of a matrix for inferring latent mutational signatures in cancer;

FIG. 4 is a plot showing an example of a signature matrix P;

FIG. 5 is a plot showing an example of mutational signatures across different cancer types in the TCGA dataset;

FIG. 6 is a plot showing an example of hierarchical clustering of individual TCGA patient samples according to their inferred mutational signature exposures;

FIG. 7 is an enlarged view of a portion of the plot of FIG. 6 showing clustering of a lung squamous cell carcinoma patient sample (TCGA-18-3409) with all of the melanoma patient samples;

FIG. 8 is a flow diagram illustrating a method for identifying somatic mutational signatures for detection of cancer, in accordance with another embodiment of the present invention;

FIG. 9 is a plot showing the estimated number of signature 1 mutations in cfDNA from cancer patients and healthy subjects as a function of age;

FIG. 10 is a bar graph showing an example of a mutational profile from a patient's cfDNA sample;

FIG. 11 is a bar graph showing the number of observed base substitution mutations of FIG. 10 for each underlying mutational signature context;

FIG. 12A is a plot showing the SNV and indel burden in cfDNA from a patient sample;

FIG. 12B is a plot showing the number of C>T base substitutions in a patient sample;

FIG. 12C is a bar graph showing the distribution of mutations with inter-mutation distance <100 bp in a patient sample and other cohort cfDNA patient samples;

FIG. 13 shows plots of sequence context and motif location relative to SNVs in sample MSK11591A;

FIG. 14 is a plot showing Signature 2;

FIG. 15 is a flow diagram illustrating a method for monitoring mutational signatures at two or more time points for the detection, diagnosis, monitoring, and/or classification of cancer, in accordance with another embodiment of the present invention;

FIG. 16 is a plot showing a simulation monitoring three mutational signatures over a plurality of time points, in accordance with the embodiment of FIG. 15;

FIGS. 17A-C are mutational count histograms determined from the aggregation of 96 trinucleotide mutational contexts to the six single base change contexts in accordance with the present invention for: (A) AID/APOBEC hypermutation; (B) cigarette smoke exposure; and (C) spontaneous deamination;

FIGS. 18A-C are mutational count histograms determined from the superposition of mutational signatures in accordance with the present invention for: (A) AID/APOBEC hypermutation at a first time point (T1); (B) AID/APOBEC hypermutation and cigarette smoke exposure at a second time point (T2); and (C) AID/APOBEC hypermutation, cigarette smoke exposure and spontaneous deamination at a third time point (T3) 15 is flowchart of a method for preparing a nucleic acid sample for sequencing according to one embodiment;

FIG. 19 is block diagram of a processing system for processing sequence reads according to one embodiment;

FIG. 20 is flowchart of a method for determining variants of sequence reads according to one embodiment;

FIG. 21 shows a different regression approach applied to a simulated mutational profile in accordance with one embodiment of the present invention;

FIG. 22 is a graph showing estimated exposure counts on the y-axis and simulated exposure counts on the x-axis. Three different regression techniques are indicated in the legend;

FIG. 23 is a bar graph showing mutation count as a function of trinucleotide context for an MSI patient for WBC and cfDNA SNVs;

FIG. 24 is a bar graph showing mutation count as a function of trinucleotide context for an MSI patient for cfDNA SNVs only;

FIG. 25 is a bar graph showing mutation count as a function of trinucleotide context for an 85 year old patient for WBC and cfDNA SNVs;

FIG. 26 is a bar graph showing mutation count as a function of trinucleotide context for an 85 year old patient for cfDNA SNVs only;

FIG. 27 is a bar graph showing mutation count as a function of trinucleotide context for a 68 year old patient for WBC and cfDNA SNVs;

FIG. 28 is a bar graph showing mutation count as a function of trinucleotide context for a 68 year old patient for cfDNA SNVs only;

FIG. 29 is a plot showing COSMIC mutational signatures 1-30 across different cancer types in the CCGA dataset;

FIG. 30 is a graph showing the proportion of each COMSIC mutational signature, divided by cancer type, across a plurality of samples;

FIG. 31 is a graph showing cfDNA fragment length distributions for three different samples for all SNVs within the samples;

FIG. 32 is a graph showing cfDNA fragment length distributions for three different samples for only T>C mutations within the samples;

FIG. 33 is a graph showing the proportion of Signature 4, divided by cancer type, and divided by smoking status.

FIG. 34 is a graph showing the proportion of Signature 6 for different cancer types, divided by cancer stage.

FIG. 35 is a graph showing indel frequency plotted as a function of Signature 6 exposure for a variety of cancer types.

FIG. 36 is a histogram of SNV and indel frequencies.

### DEFINITIONS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application, as do the following, each of which is incorporated by reference herein in its entirety: Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6th edition (Saunders, 2007).

All publications mentioned herein are expressly incorporated herein by reference to disclose and describe the methods and/or materials in connection with which the publications are cited.

The term “amplicon” as used herein means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g., “real-time PCR”, or “real-time NASBA” as described in Leone et al, Nucleic Acids Research, 26: 2150-2155 (1998), and like references.

The term “amplifying” means performing an amplification reaction. A “reaction mixture” means a solution containing all the necessary reactants for performing a reaction, which may include, but is not be limited to, buffering agents to maintain pH at a selected level during a reaction, salts, co-factors, scavengers, and the like.

The terms “fragment” or “segment”, as used interchangeably herein, refer to a portion of a larger polynucleotide molecule. A polynucleotide, for example, can be broken up, or fragmented into, a plurality of segments, either through natural processes, as is the case with, e.g., cfDNA fragments that can naturally occur within a biological sample, or through in vitro manipulation. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al, U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); the disclosures of which are hereby incorporated by reference herein in their entireties. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references, which are incorporated by reference herein in their entireties: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); and Becker-Andre et al, Nucleic Acids Research, 17: 9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2nd Edition (Cold Spring Harbor Press, New York, 2003).

The terms “subject” and “patient” are used interchangeably herein and refer to a human or non-human animal who is known to have, or potentially has, a medical condition or disorder, such as, e.g., a cancer.

The term “sequence read” as used herein refers to nucleotide sequences read from a sample obtained from a subject. Sequence reads can be obtained through various methods known in the art.

The term “read segment” or “read” as used herein refers to any nucleotide sequences, including sequence reads obtained from a subject and/or nucleotide sequences, derived from an initial sequence read from a sample. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of a nucleotide sequence, e.g., a sequence read from a sample. A substitution from a first nucleobase X to a second nucleobase Y may be denoted as “X>Y.” For example, a cytosine to thymine SNV may be denoted as “C>T.”

The term “indel” as used herein refers to any insertion or deletion of one or more base pairs having a length and a position (which may also be referred to as an anchor position) in a sequence read. An insertion corresponds to a positive length, while a deletion corresponds to a negative length.

The term “mutation” refers to one or more SNVs or indels.

The term “true positive” refers to a mutation that indicates real biology, for example, presence of a potential cancer, disease, or germline mutation in a subject. True positives are not caused by mutations naturally occurring in healthy subjects (e.g., recurrent mutations) or other sources of artifacts such as process errors during assay preparation of nucleic acid samples.

The term “false positive” refers to a mutation incorrectly determined to be a true positive. Generally, false positives may be more likely to occur when processing sequence reads associated with greater mean noise rates or greater uncertainty in noise rates.

The term “cell-free DNA” or “cfDNA” refers to nucleic acid fragments that circulate in a subject's body (e.g., bloodstream) and originate from one or more healthy cells and/or from one or more cancer cells.

The term “circulating tumor DNA” or “ctDNA” refers to nucleic acid fragments that originate from tumor cells or other types of cancer cells, which may be released into a subject's bloodstream as a result of biological processes, such as apoptosis or necrosis of dying cells, or may be actively released by viable tumor cells.

The term “alternative allele” or “ALT” refers to an allele having one or more mutations relative to a reference allele, e.g., corresponding to a known gene.

The term “sequencing depth” or “depth” refers to a total number of read segments from a sample obtained from a subject.

The term “alternate depth” or “AD” refers to a number of read segments in a sample that support an ALT, e.g., include mutations of the ALT.

The term “alternate frequency” or “AF” refers to the frequency of a given ALT. The AF may be determined by dividing the corresponding AD of a sample by the depth of the sample for the given ALT.

The term “somatic mutation” means an alteration of the DNA of a cell of a subject that occurs after conception, and which is not passed on to the subject's offspring.

The term “germline mutation” means an alteration of the DNA of a reproductive cell (e.g., a sperm or an egg cell) of a subject that becomes incorporated into the DNA of every cell in the body of the subject's offspring.

The term “somatic mutation profile” means a collection of sequence information relating to one or more somatic mutations in a subject, and that represents a quantification of variants across sequence contexts for the subject.

The term “mutational signature” means a distinguishing combination of mutations that is generated from one or more mutational processes. The term “cancer-associated mutational signature” as used herein means a mutational signature that is known to be associated with one or more specific cancers.

The term “signature matrix” means a collection of one or more individual mutational signatures that are arranged and stored on a computer-readable medium in an accessible manner.

### DETAILED DESCRIPTION OF THE INVENTION

Aspects of the invention include methods and systems for identifying somatic mutational signatures for detecting, diagnosing, monitoring and/or classifying cancer in a patient known to have, or suspected of having cancer. In various embodiments, the methods of the invention use a non-negative matrix factorization (NMF) approach to construct a signature matrix that can be used to identify latent signatures in a patient sample for detection and classification of cancer. In other embodiments, the methods of the invention may use principal components analysis (PCA) or vector quantization (VQ) approaches to construct a signature matrix. In one example, the patient sample is a cell-free nucleic acid sample (e.g., cell-free DNA (cfDNA) and/or cell-free RNA (cfRNA)).

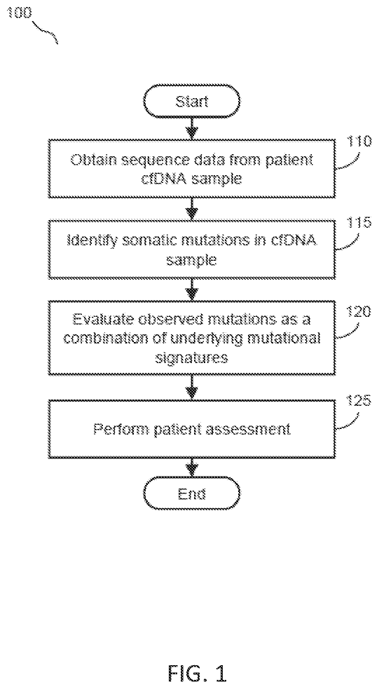

FIG. 1 illustrates a flow diagram of a method 100 for identifying somatic mutational signatures for the detection, diagnosis, monitoring, and/or classification of cancer in accordance with the present invention. Method 100 includes, but is not limited to, the following steps.

As shown in FIG. 1, at a step 110, sequencing reads are obtained from a patient test sample for identification of somatic mutations. In one embodiment, sequence reads from a test sample are aligned to a reference genome for identification of somatic mutations. In other embodiments, a de novo assembly procedure can be used for identification of somatic mutations. Sequence reads can be obtained from a patient test sample by any known means in the art. For example, in one embodiment, sequencing data or sequence reads from the cell-free DNA sample can be acquired using next generation sequencing (NGS). Next-generation sequencing methods include, for example, sequencing by synthesis technology (Illumina), pyrosequencing (454), ion semiconductor technology (Ion Torrent sequencing), single-molecule real-time sequencing (Pacific Biosciences), sequencing by ligation (SOLiD sequencing), and nanopore sequencing (Oxford Nanopore Technologies). In some embodiments, sequencing is massively parallel sequencing using sequencing-by-synthesis with reversible dye terminators. In other embodiments, sequencing is sequencing-by-ligation. In yet other embodiments, sequencing is single molecule sequencing. In still another embodiment, sequencing is paired-end sequencing. Optionally, an amplification step is performed prior to sequencing. Additional sequencing and bioinformatics methodology is described herein.

In one embodiment, a patient test sample comprising a mixture of nucleic acids contributed by cancerous cells and normal euploid (i.e., non-cancerous) cells is obtained from a subject suspected of having, or known to have, cancer. For example, the patient test sample can be a cell-free DNA sample taken from a patient's blood. In one embodiment, the sample is a plasma sample from a cancer patient. In other embodiments, the biological sample may be a sample selected from the group consisting of blood, plasma, serum, urine and saliva samples. Alternatively, the biological sample may comprise a sample selected from the group consisting of whole blood, a blood fraction, saliva/oral fluid, urine, a tissue biopsy, pleural fluid, pericardial fluid, cerebral spinal fluid, and peritoneal fluid.

At step 115, somatic mutations present in the cfDNA are identified to create an observed somatic mutational profile. In some embodiments, a mutational profile comprises a plurality of mutations identified from a patient's test sample, and can include one or more somatic mutations derived from one or more mutation signatures associated with one or more mutational processes or exposures. In some embodiments of the methods, a minimum number of SNVs is required to be present in a sample before deconvolution can be carried out. For example, in some embodiments, the methods require at least 20 SNVs to be present before deconvolution can be carried out, such as at least 25, 30, 35, 40, 45, 50, 55, 60, 65, 70, 75, 80, 85, 90, 95 or at least 100 or more SNVs. In some embodiments, the methods require that a threshold exposure proportion of a given mutational signature be present for inclusion in an analysis. For example, in some embodiments, the methods require an exposure proportion of at least 0.1, 0.15, 0.2, 0.25, 0.3, 0.35, 0.4, 0.45, 0.5, 0.55, or at least 0.6 for a given mutational signature for inclusion in an analysis.

Mutational signatures associated with one or more mutational processes are known in the art, and include, without limitation, those disclosed in Nik-Zainal S. et al., Cell (2012); Alexandrov L. B. et al., Cell Reports (2013); Alexandrov L. B. et al., Nature (2013); Helleday T. et al., Nat Rev Genet (2014); and Alexandrov L. B. and Stratton M. R., Curr Opin Genet Dev (2014), the disclosures of which are incorporated herein by reference in their entirety, and also available online at the Catalog of Somatic Mutations In Cancer (COSMIC) website, at http://cancer.sanger.ac.uk/cosmic/signatures. The analysis reported on the COSMIC website utilizes 30 known mutational signatures, and 96 trinucleotide sequence contexts. The methods described herein are not limited to the 30 mutational signatures or the 96 trinucleotide sequence contexts reported on the COSMIC website, but these are merely provided as examples. Those of ordinary skill in the art will readily appreciate that other mutational signatures and/or sequence contexts can be utilized in conjunction with the methods described herein.

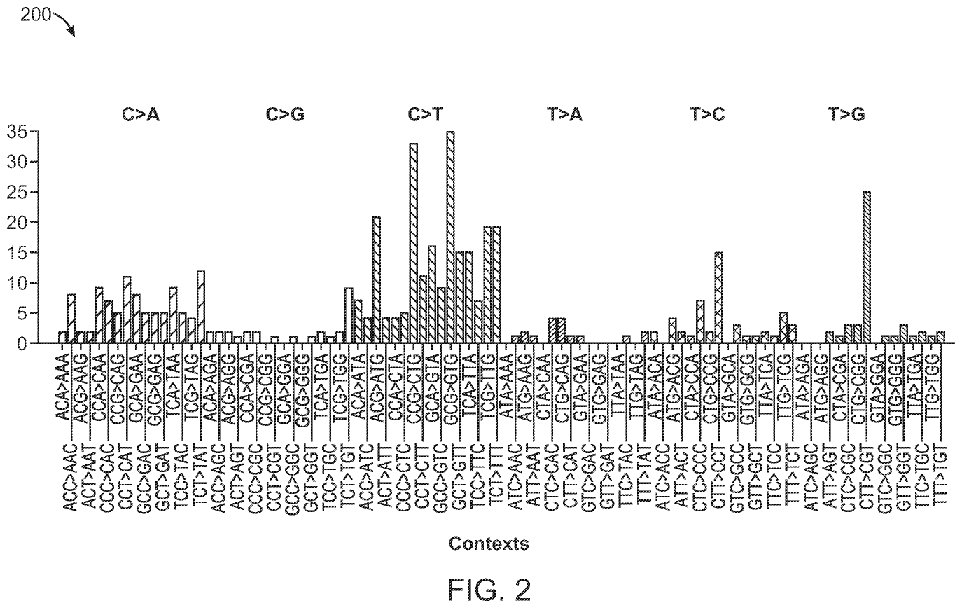

In one embodiment, an observed mutational profile can include sequence context of base substitutions in the patient's cfDNA as described in more detail with reference to FIG. 2.

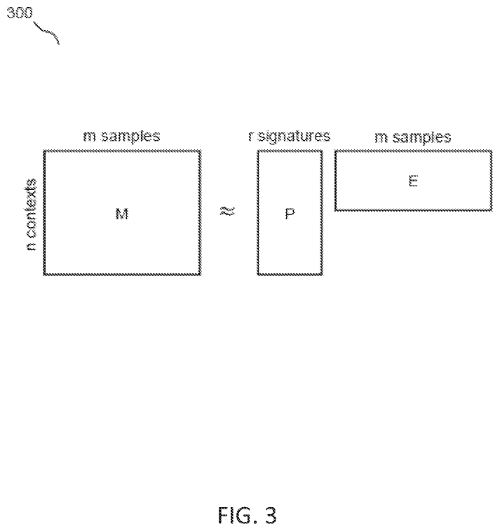

At a step 120, the observed mutational profile in cfDNA from the patient sample is evaluated as a combination of different mutational signatures represented in a signature matrix P. Signature matrix P is a representation of underlying mutational signatures identified in a training set. For example, in one embodiment, signature matrix P is a representation of mutational signatures identified for, or derived from, a number of mutational profiles from cancer patient samples with known cancer status across different cancer types. As used herein, the term “cancer status” refers to the presence or absence of cancer, stage of cancer, the cancer cell-type, and/or the cancer tissue of origin. In accordance with this embodiment, signature matrix P represents a plurality of unique mutational signatures associated with different mutational processes from cancer patient samples with known cancer status. The construction of a signature matrix P is described in more detail with reference to FIG. 3.

At a step 125, an assessment of the patient's cancer status is inferred from the patient's unique mutational profile through inferring the latent exposure weights contributed by each mutational signature. This inference can be framed as inference on a mixture model or mathematical optimization. For example, in one embodiment, non-negative linear regression can be used to determine, or infer, cancer status from the patient's unique mutational profile. Another example, would be to apply nonlinear optimization to maximize orthogonality between the signature exposure weights. In another embodiment, a cancer cell-type or tissue of origin can be inferred from the patient's unique mutational profile through inferring the latent exposure weights contributed by one or more mutational signature. In still another embodiment, one or more causative mutational process can be inferred from the patient's unique mutational profile through inferring the latent exposure weights contributed by one or more of the mutational signatures.

FIG. 2 is a bar graph 200 showing an example mutational profile determined from sequencing data obtained from a patient test sample. In accordance with this embodiment, the identified somatic mutations, and thus, the mutational profile, are conditioned on triplet sequence context of base substitution mutations identified in the patient's test sample. There are about 400 mutations in this patient sample. In this example, the mutational profile comprises the frequency of mutations identified for each sequence context and is displayed based on the six base substitution subtypes identified: C>A, C>G, C>T, T>A, T>C, and T>G. As shown in FIG. 2, there are approximately 400 identified mutations within 16 possible sequence contexts for each of the 6 base substitution subtypes identified. Because there are six subtypes of base substitutions and 16 possible sequence context for each mutated base there are 96 possible trinucleotide contexts. The sequence context of each mutation is recorded and the frequency of each mutation in each context is calculated.

Application of non-negative matrix factorization to infer latent mutational signatures for cancer detection, diagnosis and classification.

In accordance with the present invention, a machine learning approach can be utilized to infer underlying mutational signatures identified in a patient test sample (e.g., a cell-free nucleic acid sample). In general, any known machine learning approach can be utilized in practicing the present invention. For example, in one embodiment, non-negative matrix factorization can be utilized as a machine learning approach to decompose, or deconvolute, an observed matrix and identify underlying signatures prevalent in the dataset. To infer underlying mutational signatures we decompose a matrix constructed of patient samples to explain the observed mutational frequency contexts as a combination of the underlying mutational signatures (i.e., r mutational signatures) and the exposure each patient has to those r mutational signatures (i.e., E exposure weights). In another embodiment, principal components analysis or vector quantization can be used.

FIG. 3 illustrates a schematic diagram of a process 300 of inferring latent mutational signatures in cancer, in accordance with one embodiment of the present invention. As shown in FIG. 3, sample matrix “M” is a dataset made up of 96 features (n contexts; represented in rows) comprising counts for each mutation type identified (C>A, C>G, C>T, T>A, T>C, and T>G) from m number of cancer patient samples (m samples; represented in columns). In one embodiment, sample matrix M can be constructed from about 50 or more patient samples. In other embodiments, sample matrix M can comprise more than 100, more than 1,000, more than 10,000, or more than 100,000 mutational profiles from cancer patients. In other embodiments, sample matrix M can comprise from about 10 to more than 1 million, from about 10 to about 100,000, from about 50 to about 10,000, from about 100 to about 1,000 mutational profiles identified from cancer patients. As described in more detail above, FIG. 2 provides an example of a single patient mutational profile, which represents one column in sample matrix M.

As shown in FIG. 3, sample matrix M can be decomposed, or deconvoluted, using non-negative matrix factorization into two nonnegative matrices: a matrix “P” of r number of mutational signatures by n contexts (or features) (where elements of P take values in [0, 1]) and a matrix “E” of exposure weights that each patient has to the r mutational signatures. The product of signature matrix P and exposure matrix E (P×E) for a patient sample is an approximate reconstruction of the observed mutations for a given patient test sample. As described above, examples of relevant features (or n contexts) include, but are not limited to, an upstream sequence context of a base substitution mutation, a downstream sequence context of base a substitution mutations, an insertion, a deletion, a somatic copy number alteration (SCNA), a translocation, a genomic methylation status, a chromatin state, a sequencing depth of coverage, an early versus late replicating region, a sense versus antisense strand, an inter mutation distance, a variant allele frequency, a fragment start/stop, a fragment length, and a gene expression status, or any combination thereof.

Accordingly, in the practice of the present invention, non-negative matrix factorization can be used to reconstruct latent mutational signatures (i.e., r number of mutational signatures) that underlie mutational profiles (i.e., mutation frequency contexts) in cancer patient samples. In the context of cancer detection, diagnosis, or classification, reconstruction of the latent mutational signatures including their exposure weights observed for a new patient test sample can be used to infer the presence or absence of cancer, or cancer status. This approach allows biological interpretations (e.g., signatures of known mutational processes such as arising from endogenous or exogenous DNA damage, DNA modification, DNA editing, DNA repair, DNA replication) to be superimposed on an observed mutational profile from a new patient test sample.

The construction of signature matrix P is an iterative process. For example, an existing dataset of somatic mutation data can be used to build, or construct, matrix M comprising mutational context for m number of known cancer data sets. The matrix M can then be used to construct signature matrix P using non-negative matrix factorization and applied to infer, or determine, cancer status for an unknown test sample based on the underlying mutational signature observed for a new patient test sample. In one example, the mutation dataset can be built, or constructed from, sequencing data available for known cancers through The Cancer Genome Atlas (TCGA), International Cancer Genome Consortium (ICGC), or other publicly available data bases. In one embodiment, as additional sequencing data is obtained for new patient test samples (e.g., from cfDNA), sample matrix M can be updated with the new data and the performance of signature matrix P can be re-evaluated, or a new P can be generated. The process can be repeated any number of times to construct a matrix for optimal (robust) performance. It is believed that signature matrix P improves as sample size increases as subsampling analysis of a patient cohort has demonstrated that the performance of non-negative matrix factorization decreases with sample size (data not shown). The decrease in performance with decreased sample size can also be demonstrated using simulation models (data not shown). Once a robust signature matrix P is constructed, the completed signature matrix P can be used alone (i.e., without non-negative matrix factorization) to assess new patient samples.

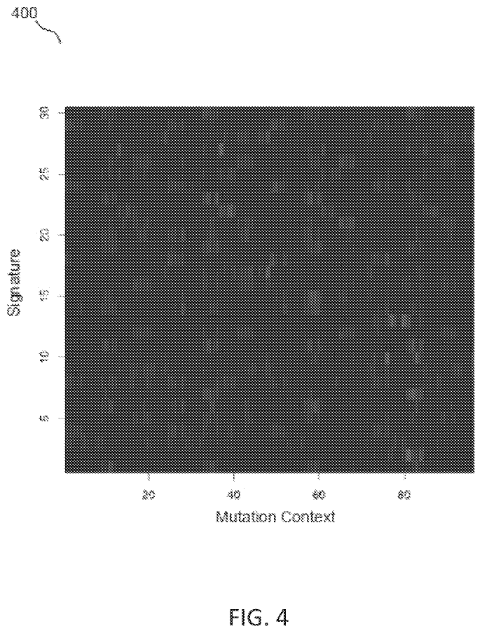

FIG. 4 is a plot 400 showing an example signature matrix P constructed using non-negative factorization, in accordance with the present invention. The elements of signature matrix P are mutational signatures derived from the sample matrix M. As shown in FIG. 4, 30 mutational signatures are represented in combination with mutational context. Each mutational signature is characterized by a different profile of the 96 trinucleotide mutation contexts.

In other embodiments, in addition to sequence context (e.g., triplet sequence context) of base substitutions as described herein, non-negative matrix factorization can be applied to somatic copy number alterations (SCNA), genomic methylation status, and/or gene transcription (e.g., analyzing cell-free RNA).

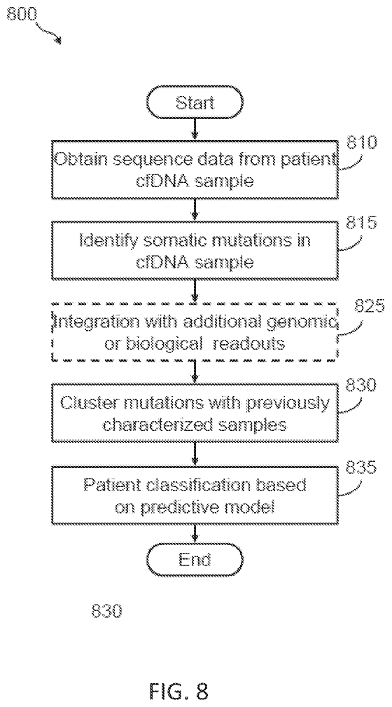

FIG. 8 is a flow diagram illustrating a method 800 for identifying somatic mutational signatures for the detection, diagnosis, monitoring, and/or classification of cancer in accordance with another embodiment of the present invention. As shown in FIG. 8, method 800 may include, but is not limited to, the following steps.

At step 810, sequencing reads are obtained from a patient test sample and used for identification of somatic mutations. In one embodiment, sequence reads from a test sample are aligned to a reference genome for identification of somatic mutations. In another embodiment, a de novo assembly procedure can be used for identification of somatic mutations. As discussed in more detail herein, sequence reads can be obtained from a patient test sample by any suitable means. Also, as noted herein, a patient test sample can comprise a mixture of nucleic acids contributed by cancerous cells and normal euploid (i.e., non-cancerous) cells obtained from a subject suspected of having, or known to have, cancer. For example, in some embodiments, a patient test sample can be a cell-free DNA sample taken from a patient's blood.

At step 815, somatic mutations present in the cfDNA are identified to create an observed somatic mutational profile. In one embodiment, the observed mutational profile can include sequence context of base substitutions in the patient's cfDNA as described in more detail with reference to FIG. 2.

Optionally, at step 825, the clustered mutation profiles can be integrated with additional genomic or biological data. For example, one or more functional annotations can be used for classification of a patient specific sample. The one or more functional annotations can include, but are not limited to, spatial clustering within a signature class between and within subjects, statistical association with chromatin state that differs systemically between tissues, statistical association with early versus late replicating regions (e.g., replication associated repair), statistical association with expression or strandedness (e.g., defects related to transcription coupled repair), statistical association with germline variants/somatic variants and somatic signatures (e.g., loss of proofreading function mutations in polymerase ε or polymerase 6), or stratification according to fragment length.

At step 830, the observed mutational profile can be clustered (e.g., using a clustering procedure) with other mutational signatures identified from previously characterized samples.

At step 835, a patient specific classification is determined based on the patient's unique mutational profile. For example, in some embodiments, an assessment of the patient's cancer status can be inferred from the patient's mutational profile through inferring the latent exposure weights contributed by each mutational signature. This inference can be framed as inference on a mixture model or mathematical optimization. For example, in one embodiment, non-negative linear regression can be used to determine, or infer, cancer status from the patient's unique mutational profile and a matrix of mutational signatures. In some embodiments, a nonlinear optimization protocol can be applied to maximize orthogonality between the inferred combination mutational signature. In another embodiment, a cancer cell-type or tissue of origin can be inferred from the patient's unique mutational profile through inferring the latent exposure weights contributed by one or more mutational signatures. In still another embodiment, one or more causative mutational process can be inferred from the patient's unique mutational profile through inferring the latent exposure weights contributed by one or more mutational signatures.

In another embodiment, non-negative matrix factorization can be applied to learn error modes in a somatic variant calling assay. The process of non-negative matrix factorization does not make assumptions about the underlying biology of a variant. Systematic errors (e.g., errors contributed during library preparation, PCR, hybridization capture, and/or sequencing) that underlie the assay can be identified, and assigned unique signatures that can be used to distinguish between the contribution from true somatic mutations and artifactual mutations arising from the technical processes in the assay. The learned error signatures can then be accounted for in the analysis of somatic mutation candidates to reduce false positive calls.

In yet another embodiment, non-negative matrix factorization can be used to account for somatic mutation(s) associated with healthy aging. It is known that the cumulative contribution of certain mutation processes (e.g., the spontaneous deamination of 5-methylcytosine) are associated with the number of cell divisions. Each process can be associated with a mutational signature that can be used to distinguish between healthy somatic mutation(s) associated with patient age and somatic mutation(s) contributed from a cancer process in the patient.

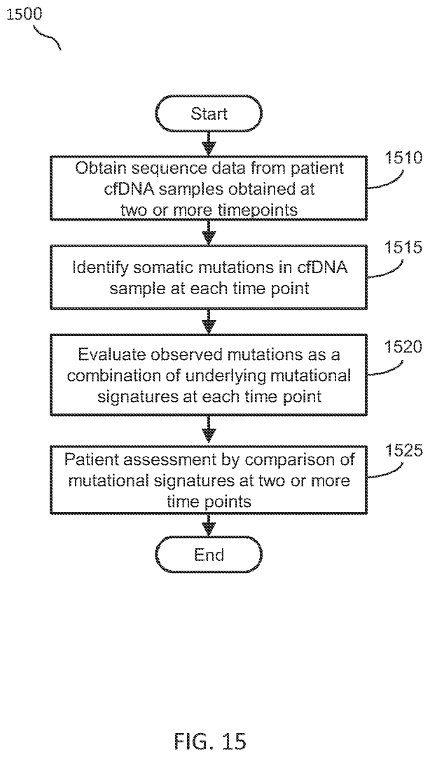

FIG. 15 illustrates a flow diagram of a method 1500 for monitoring mutational signatures for the detection, diagnosis, monitoring, and/or classification of cancer in accordance with the present invention. Method 1500 includes, but is not limited to, the following steps.

As shown in FIG. 15, at a step 1510, sequencing reads are obtained from test samples obtained from a patient at two or more time points (e.g., a first time point and a second time point) and used for identification of one or more mutational signatures. As described above, sequence reads or sequencing data can be obtained using any known means in the art, and sequence reads aligned to a reference genome, or used for de novo assembly, for identification of one or more somatic mutations. As described elsewhere, the somatic mutations can be used to determine a mutational profile, or to identify a mutational signature, at each of the time points. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

At a step 1515, somatic mutations present in the cfDNA at each of the two or more time points are identified to create an observed somatic mutational profile, or to identify mutational signatures, for each time point. As previously described, the term mutational profile may comprise a collection of one or more mutations in a test sample from a patient. In some embodiments, the mutational profile comprises a plurality of mutations identified from a patient's test sample, and can include one or more somatic mutations derived from one or more mutation signatures associated with one or more mutational processes or exposures. In one embodiment, the observed mutational profile can include sequence context of base substitutions in the patient's cfDNA as described in more detail with reference to FIG. 2.

At a step 1520, the observed mutational profile, and/or mutational signatures, in the patient test samples obtained at two or more time points are evaluated. In some embodiments, the mutational profiles obtained at each time point may comprise a combination of different mutational signature processes. For example, the mutational profile at each time point may comprise a combination of two or more mutational profiles determined for two or more known mutational processes (e.g., two or more known COSMIC mutational processes). In other embodiments, mutational profiles, or a combination of mutational profiles from two or more mutational processes can be identified from each of the test samples and monitored over time.

At a step 1525, an assessment of the patient's cancer status is determined, or monitored, by comparison of mutational signatures determined from patient test samples obtained at two or more time points. For example, the patient's unique mutational profile can be determined at two or more time points through inferring the latent exposure weights contributed by each mutational signature at each time point. As previously described, this inference can be framed as inference on a mixture model or mathematical optimization. In still other embodiments, one or more mutational signatures can be monitored over time (e.g., at a plurality of time points) to monitor the effectiveness of a therapeutic regimen or other cancer treatment.

### Example Assay Protocol

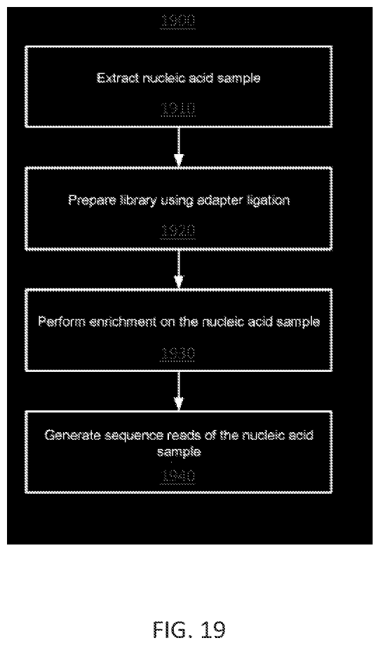

FIG. 19 is flowchart of a non-limiting example of a method 1900 for preparing a nucleic acid sample for sequencing according to one embodiment. The method 1900 includes, but is not limited to, the following steps. For example, any step of the method 1900 may comprise a quantitation sub-step for quality control or other laboratory assay procedures known to one skilled in the art.

In step 1910, a nucleic acid sample (DNA or RNA) is extracted from a subject. In the present disclosure, DNA and RNA may be used interchangeably unless otherwise indicated. That is, the following embodiments for using error source information in variant calling and quality control may be applicable to both DNA and RNA types of nucleic acid sequences. However, the examples described herein may focus on DNA for purposes of clarity and explanation. The sample can comprise any subset of the human genome, including the whole genome. The sample may be extracted from a subject known to have or suspected of having cancer. The sample may include a tissue, a body fluid, or a combination thereof, as described further herein. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has a cancer or disease, ctDNA in an extracted sample may be present at a detectable level for diagnosis.

In step 1920, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In step 1930, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer cell-type or tissue of origin). For a given workflow, the probes may be designed to anneal (or hybridize) to a target (complementary) strand of DNA or RNA. The target strand may be the “positive” strand (e.g., the strand transcribed into mRNA, and subsequently translated into a protein) or the complementary “negative” strand. The probes may range in length from 10s, 100s, or 1000s of base pairs. In one embodiment, the probes are designed based on a gene panel to analyze particular mutations or target regions of the genome (e.g., of the human or another organism) that are suspected to correspond to certain cancers or other types of diseases. Moreover, the probes may cover overlapping portions of a target region. By using a targeted gene panel rather than sequencing all expressed genes of a genome, also known as “whole exome sequencing,” the method 100 may be used to increase sequencing depth of the target regions, where depth refers to the count of the number of times a given target sequence within the sample has been sequenced. Increasing sequencing depth reduces required input amounts of the nucleic acid sample. After a hybridization step, the hybridized nucleic acid fragments are captured and may also be amplified using PCR.

In step 1940, sequence reads are generated from the enriched DNA sequences. Sequencing data may be acquired from the enriched DNA sequences by known means in the art. For example, the method 1900 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R1 and R2. For example, the first read R1 may be sequenced from a first end of a nucleic acid fragment whereas the second read R2 may be sequenced from the second end of the nucleic acid fragment. Therefore, nucleotide base pairs of the first read R1 and second read R2 may be aligned consistently (e.g., in opposite orientations) with nucleotide bases of the reference genome. Alignment position information derived from the read pair R1 and R2 may include a beginning position in the reference genome that corresponds to an end of a first read (e.g., R1) and an end position in the reference genome that corresponds to an end of a second read (e.g., R2). In other words, the beginning position and end position in the reference genome represent the likely location within the reference genome to which the nucleic acid fragment corresponds. An output file having SAM (sequence alignment map) format or BAM (binary) format may be generated and output for further analysis such as variant calling, as described below with respect to FIG. 19.

### Example Processing System

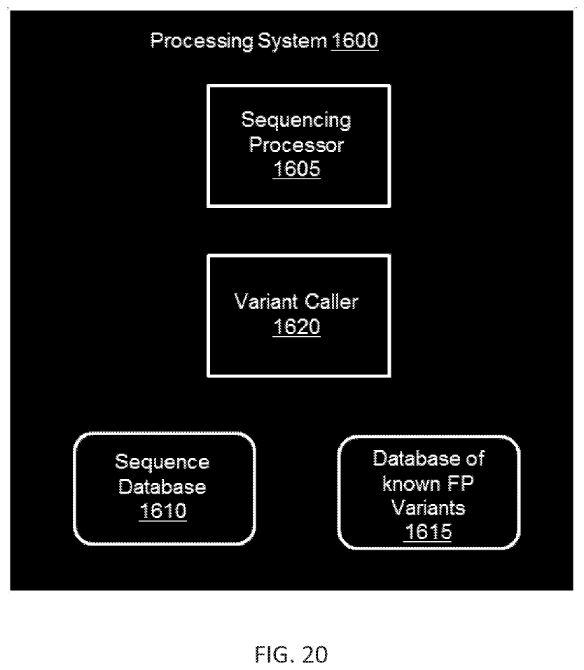

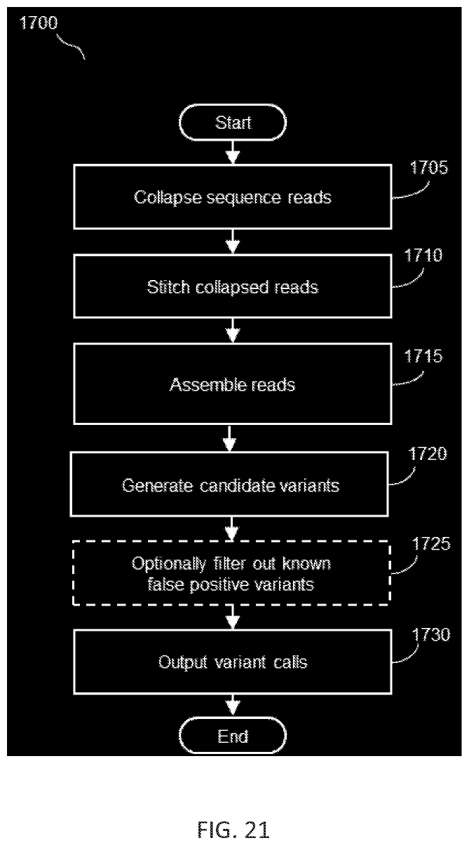

FIG. 20 is block diagram of a processing system 1600 for processing sequence reads according to one embodiment. The processing system 1600 includes a sequence processor 1605, sequence database 1610, a database of known true positive (TP) and false positive (FP) variants 1615, and variant caller 1620. FIG. 21 is flowchart of a method 1700 for determining variants of sequence reads according to one embodiment. In some embodiments, the processing system 1600 performs the method 1700 to perform variant calling (e.g., for SNVs and/or indels) based on input sequencing data. Further, the processing system 1600 may obtain the input sequencing data from an output file associated with nucleic acid sample prepared using the method 1500 described above. The method 1700 includes, but is not limited to, the following steps, which are described with respect to the components of the processing system 1600. In other embodiments, one or more steps of the method 1700 may be replaced by a step of a different process for generating variant calls, e.g., using Variant Call Format (VCF), such as HaplotypeCaller, VarScan, Strelka, or SomaticSniper.

At step 1705, the sequence processor 1605 collapses aligned sequence reads of the input sequencing data. In one embodiment, collapsing sequence reads includes using UMIs, and optionally alignment position information from sequencing data of an output file (e.g., from the method 1500 shown in FIG. 19) to collapse multiple sequence reads into a consensus sequence for determining the most likely sequence of a nucleic acid fragment or a portion thereof. Since the UMIs are replicated with the ligated nucleic acid fragments through enrichment and PCR, the sequence processor 1605 may determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor 1605 generates a collapsed read (also referred to herein as a consensus read) to represent the nucleic acid fragment. The sequence processor 1605 designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the sequence processor 1605 may perform other types of error correction on sequence reads as an alternate to, or in addition to, collapsing sequence reads.

At step 1710, the sequence processor 1605 stitches the collapsed reads based on the corresponding alignment position information. In some embodiments, the sequence processor 1605 compares alignment position information between a first read and a second read to determine whether nucleotide base pairs of the first and second reads overlap in the reference genome. In one use case, responsive to determining that an overlap (e.g., of a given number of nucleotide bases) between the first and second reads is greater than a threshold length (e.g., threshold number of nucleotide bases), the sequence processor 1605 designates the first and second reads as “stitched”; otherwise, the collapsed reads are designated “unstitched.” In some embodiments, a first and second read are stitched if the overlap is greater than the threshold length and if the overlap is not a sliding overlap. For example, a sliding overlap may include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide run, or trinucleotide run has at least a threshold length of base pairs.

At step 1715, the sequence processor 1605 assembles reads into paths. In some embodiments, the sequence processor 1605 assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The sequence processor 1605 aligns collapsed reads to a directed graph such that any of the collapsed reads may be represented in order by a subset of the edges and corresponding vertices.

In some embodiments, the sequence processor 1605 determines sets of parameters describing directed graphs and processes directed graphs. Additionally, the set of parameters may include a count of successfully aligned k-mers from collapsed reads to a k-mer represented by a node or edge in the directed graph. The sequence processor 1605 stores, e.g., in the sequence database 1610, directed graphs and corresponding sets of parameters, which may be retrieved to update graphs or generate new graphs. For instance, the sequence processor 1605 may generate a compressed version of a directed graph (e.g., or modify an existing graph) based on the set of parameters. In one use case, in order to filter out data of a directed graph having lower levels of importance, the sequence processor 1605 removes (e.g., “trims” or “prunes”) nodes or edges having a count less than a threshold value, and maintains nodes or edges having counts greater than or equal to the threshold value.

At step 1720, the variant caller 1620 generates candidate variants from the paths assembled by the sequence processor 1605. In one embodiment, the variant caller 1620 generates the candidate variants by comparing a directed graph (which may have been compressed by pruning edges or nodes in step 1715) to a reference sequence of a target region of a genome. The variant caller 1620 may align edges of the directed graph to the reference sequence, and records the genomic positions of mismatched edges and mismatched nucleotide bases adjacent to the edges as the locations of candidate variants. Additionally, the variant caller 1620 may generate candidate variants based on the sequencing depth of a target region. In particular, the variant caller 1620 may be more confident in identifying variants in target regions that have greater sequencing depth, for example, because a greater number of sequence reads help to resolve (e.g., using redundancies) mismatches or other base pair variations between sequences.

At step 1725, the processing system 1600 outputs the candidate variants. In some embodiments, the processing system 1600 outputs some or all of the determined candidate variants. In other embodiments, optionally, the candidate variants can be filtered to remove known false positive variants. For example, the candidate variants can be compared with known false positive variants, the false positive variants, and filtered variant calls output. Downstream systems, e.g., external to the processing system 1600 or other components of the processing system 1600, may use the candidate variants for various applications including, but not limited to, predicting presence of cancer, disease, or germline mutations.

### Sequencing and Bioinformatics

Aspects of the invention include sequencing of nucleic acid molecules to generate a plurality of sequence reads, and bioinformatic manipulation of the sequence reads to carry out the subject methods.

In certain embodiments, a sample is collected from a subject, followed by enrichment for genetic regions or genetic fragments of interest. For example, in some embodiments, a sample can be enriched by hybridization to a nucleotide array comprising cancer-related genes or gene fragments of interest. In some embodiments, a sample can be enriched for genes of interest (e.g., cancer-associated genes) using other methods known in the art, such as hybrid capture. See, e.g., Lapidus (U.S. Pat. No. 7,666,593), the contents of which is incorporated by reference herein in its entirety. In one hybrid capture method, a solution-based hybridization method is used that includes the use of biotinylated oligonucleotides and streptavidin coated magnetic beads. See, e.g., Duncavage et al., J Mol Diagn. 13(3): 325-333 (2011); and Newman et al., Nat Med. 20(5): 548-554 (2014). Isolation of nucleic acid from a sample in accordance with the methods of the invention can be done according to any method known in the art.

Sequencing may be by any method or combination of methods known in the art. For example, known DNA sequencing techniques include, but are not limited to, classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, Polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977), the contents of which are incorporated by reference herein in their entirety. Another conventional sequencing method involves chemical degradation of nucleic acid fragments. See, Maxam et al., Proc. Natl. Acad. Sci., 74: 560 564 (1977), the contents of which are incorporated by reference herein in their entirety. Methods have also been developed based upon sequencing by hybridization. See, e.g., Harris et al., (U.S. patent application number 2009/0156412), the contents of which are incorporated by reference herein in their entirety.

A sequencing technique that can be used in the methods of the provided invention includes, for example, Helicos True Single Molecule Sequencing (tSMS) (Harris T. D. et al. (2008) Science 320:106-109), the contents of which are incorporated by reference herein in their entirety. Further description of tSMS is shown, for example, in Lapidus et al. (U.S. Pat. No. 7,169,560), the contents of which are incorporated by reference herein in their entirety, Lapidus et al. (U.S. patent application publication number 2009/0191565, the contents of which are incorporated by reference herein in their entirety), Quake et al. (U.S. Pat. No. 6,818,395, the contents of which are incorporated by reference herein in their entirety), Harris (U.S. Pat. No. 7,282,337, the contents of which are incorporated by reference herein in their entirety), Quake et al. (U.S. patent application publication number 2002/0164629, the contents of which are incorporated by reference herein in their entirety), and Braslaysky, et al., PNAS (USA), 100: 3960-3964 (2003), the contents of which are incorporated by reference herein in their entirety.

Another example of a DNA sequencing technique that can be used in the methods of the provided invention is 454 sequencing (Roche) (Margulies, M et al. 2005, Nature, 437, 376-380, the contents of which are incorporated by reference herein in their entirety). Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application publication numbers 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the contents of each of which are incorporated by reference herein in their entirety).

In some embodiments, the sequencing technology is Illumina sequencing. Illumina sequencing is based on the amplification of DNA on a solid surface using fold-back PCR and anchored primers. Genomic DNA can be fragmented, or in the case of cfDNA, fragmentation is not needed due to the already short fragments. Adapters are ligated to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. Yet another example of a sequencing technique that can be used in the methods of the provided invention is nanopore sequencing (Soni G V and Meller A. (2007) Clin Chem 53: 1996-2001, the contents of which are incorporated by reference herein in their entirety). Another example of a sequencing technique that can be used in the methods of the provided invention involves using a chemical-sensitive field effect transistor (chemFET) array to sequence DNA (for example, as described in US Patent Application Publication No. 20090026082, the contents of which are incorporated by reference herein in their entirety). Another example of a sequencing technique that can be used in the methods of the provided invention involves using an electron microscope (Moudrianakis E. N. and Beer M. Proc Natl Acad Sci USA. 1965 March; 53:564-71, the contents of which are incorporated by reference herein in their entirety).

If the nucleic acid from the sample is degraded or only a minimal amount of nucleic acid can be obtained from the sample, PCR can be performed on the nucleic acid in order to obtain a sufficient amount of nucleic acid for sequencing (See, e.g., Mullis et al. U.S. Pat. No. 4,683,195, the contents of which are incorporated by reference herein in its entirety).

### Biological Samples

Aspects of the invention involve obtaining a sample, e.g., a biological sample, such as a tissue and/or body fluid sample, from a subject for purposes of analyzing a plurality of nucleic acids (e.g., a plurality of cfDNA molecules) therein. Samples in accordance with embodiments of the invention can be collected in any clinically-acceptable manner. Any sample suspected of containing a plurality of nucleic acids can be used in conjunction with the methods of the present invention. In some embodiments, a sample can comprise a tissue, a body fluid, or a combination thereof. In some embodiments, a biological sample is collected from a healthy subject. In some embodiments, a biological sample is collected from a subject who is known to have a particular disease or disorder (e.g., a particular cancer or tumor). In some embodiments, a biological sample is collected from a subject who is suspected of having a particular disease or disorder.

As used herein, the term “tissue” refers to a mass of connected cells and/or extracellular matrix material(s). Non-limiting examples of tissues that are commonly used in conjunction with the present methods include skin, hair, finger nails, endometrial tissue, nasal passage tissue, central nervous system (CNS) tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or non-human mammal. Tissue samples in accordance with embodiments of the invention can be prepared and provided in the form of any tissue sample types known in the art, such as, for example and without limitation, formalin-fixed paraffin-embedded (FFPE), fresh, and fresh frozen (FF) tissue samples.

As used herein, the term “body fluid” refers to a liquid material derived from a subject, e.g., a human or non-human mammal. Non-limiting examples of body fluids that are commonly used in conjunction with the present methods include mucous, blood, plasma, serum, serum derivatives, synovial fluid, lymphatic fluid, bile, phlegm, saliva, sweat, tears, sputum, amniotic fluid, menstrual fluid, vaginal fluid, semen, urine, cerebrospinal fluid (CSF), such as lumbar or ventricular CSF, gastric fluid, a liquid sample comprising one or more material(s) derived from a nasal, throat, or buccal swab, a liquid sample comprising one or more materials derived from a lavage procedure, such as a peritoneal, gastric, thoracic, or ductal lavage procedure, and the like.

In some embodiments, a sample can comprise a fine needle aspirate or biopsied tissue. In some embodiments, a sample can comprise media containing cells or biological material. In some embodiments, a sample can comprise a blood clot, for example, a blood clot that has been obtained from whole blood after the serum has been removed. In some embodiments, a sample can comprise stool. In one preferred embodiment, a sample is drawn whole blood. In one aspect, only a portion of a whole blood sample is used, such as plasma, red blood cells, white blood cells, and platelets. In some embodiments, a sample is separated into two or more component parts in conjunction with the present methods. For example, in some embodiments, a whole blood sample is separated into plasma, red blood cell, white blood cell, and platelet components.

In some embodiments, a sample includes a plurality of nucleic acids not only from the subject from which the sample was taken, but also from one or more other organisms, such as viral DNA/RNA that is present within the subject at the time of sampling.

Nucleic acid can be extracted from a sample according to any suitable methods known in the art, and the extracted nucleic acid can be utilized in conjunction with the methods described herein. See, e.g., Maniatis, et al., Molecular Cloning: A Laboratory Manual, Cold Spring Harbor, N.Y., pp. 280-281, 1982, the contents of which are incorporated by reference herein in their entirety.

In one preferred embodiment, cell free nucleic acid (e.g., cfDNA) is extracted from a sample. cfDNA are short base nuclear-derived DNA fragments present in several bodily fluids (e.g. plasma, stool, urine). See, e.g., Mouliere and Rosenfeld, PNAS 112(11): 3178-3179 (March 2015); Jiang et al., PNAS (March 2015); and Mouliere et al., Mol Oncol, 8(5):927-41 (2014). Tumor-derived circulating tumor DNA (ctDNA) constitutes a minority population of cfDNA, in some cases, varying up to about 50%. In some embodiments, ctDNA varies depending on tumor stage and tumor type. In some embodiments, ctDNA varies from about 0.001% up to about 30%, such as about 0.01% up to about 20%, such as about 0.01% up to about 10%. The covariates of ctDNA are not fully understood, but appear to be positively correlated with tumor type, tumor size, and tumor stage. E.g., Bettegowda et al, Sci Trans Med, 2014; Newmann et al, Nat Med, 2014. Despite the challenges associated with the low population of ctDNA in cfDNA, tumor variants have been identified in ctDNA across a wide span of cancers. E.g., Bettegowda et al, Sci Trans Med, 2014. Furthermore, analysis of cfDNA versus tumor biopsy is less invasive, and methods for analyzing, such as sequencing, enable the identification of sub-clonal heterogeneity. Analysis of cfDNA has also been shown to provide for more uniform genome-wide sequencing coverage as compared to tumor tissue biopsies. In some embodiments, a plurality of cfDNA is extracted from a sample in a manner that reduces or eliminates co-mingling of cfDNA and genomic DNA. For example, in some embodiments, a sample is processed to isolate a plurality of the cfDNA therein in less than about 2 hours, such as less than about 1.5, 1 or 0.5 hours.

A non-limiting example of a procedure for preparing nucleic acid from a blood sample follows. Blood may be collected in 10 mL EDTA tubes (for example, the BD VACUTAINER® family of products from Becton Dickinson, Franklin Lakes, N.J.), or in collection tubes that are adapted for isolation of cfDNA (for example, the CELL FREE DNA BCT® family of products from Streck, Inc., Omaha, Nebr.) can be used to minimize contamination through chemical fixation of nucleated cells, but little contamination from genomic DNA is observed when samples are processed within 2 hours or less, as is the case in some embodiments of the present methods. Beginning with a blood sample, plasma may be extracted by centrifugation, e.g., at 3000 rpm for 10 minutes at room temperature minus brake. Plasma may then be transferred to 1.5 ml tubes in 1 ml aliquots and centrifuged again at 7000 rpm for 10 minutes at room temperature. Supernatants can then be transferred to new 1.5 ml tubes. At this stage, samples can be stored at −80° C. In certain embodiments, samples can be stored at the plasma stage for later processing, as plasma may be more stable than storing extracted cfDNA.

Plasma DNA can be extracted using any suitable technique. For example, in some embodiments, plasma DNA can be extracted using one or more commercially available assays, for example, the QIAmp Circulating Nucleic Acid Kit family of products (Qiagen N.V., Venlo Netherlands). In certain embodiments, the following modified elution strategy may be used. DNA may be extracted using, e.g., a QIAmp Circulating Nucleic Acid Kit, following the manufacturer's instructions (maximum amount of plasma allowed per column is 5 mL). If cfDNA is being extracted from plasma where the blood was collected in Streck tubes, the reaction time with proteinase K may be doubled from 30 min to 60 min. Preferably, as large a volume as possible should be used (i.e., 5 mL). In various embodiments, a two-step elution may be used to maximize cfDNA yield. First, DNA can be eluted using 300 μL of buffer AVE for each column. A minimal amount of buffer necessary to completely cover the membrane can be used in the elution in order to increase cfDNA concentration. By decreasing dilution with a small amount of buffer, downstream desiccation of samples can be avoided to prevent melting of double stranded DNA or material loss. Subsequently, about 300 μL of buffer for each column can be eluted. In some embodiments, a second elution may be used to increase DNA yield.

### Computer Systems and Devices

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer programs include, by way of example, both general and special purpose microprocessors, and any one or more processors of any kind of digital computer. Generally, a processor will receive instructions and data from a read-only memory or a random access memory, or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of nonvolatile memory, including, by way of example, semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through a network by any form or medium of digital data communication, e.g., a communication network. For example, a reference set of data may be stored at a remote location and a computer can communicate across a network to access the reference data set for comparison purposes. In other embodiments, however, a reference data set can be stored locally within the computer, and the computer accesses the reference data set within the CPU for comparison purposes. Examples of communication networks include, but are not limited to, cell networks (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, a data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification (RFID) tags or chips, or any other medium that can be used to store the desired information, and which can be accessed by a computing device.

Functions described herein can be implemented using software, hardware, firmware, hardwiring, or combinations of any of these. Any of the software can be physically located at various positions, including being distributed such that portions of the functions are implemented at different physical locations.

As one skilled in the art would recognize as necessary or best-suited for performance of the methods of the invention, a computer system for implementing some or all of the described inventive methods can include one or more processors (e.g., a central processing unit (CPU) a graphics processing unit (GPU), or both), main memory and static memory, which communicate with each other via a bus.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc.

While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to, one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

Additionally, systems of the invention can be provided to include reference data. Any suitable genomic data may be stored for use within the system. Examples include, but are not limited to: comprehensive, multi-dimensional maps of the key genomic changes in major types and subtypes of cancer from The Cancer Genome Atlas (TCGA); a catalog of genomic abnormalities from The International Cancer Genome Consortium (ICGC); a catalog of somatic mutations in cancer from COSMIC; the latest builds of the human genome and other popular model organisms; up-to-date reference SNPs from dbSNP; gold standard indels from the 1000 Genomes Project and the Broad Institute; exome capture kit annotations from Illumina, Agilent, Nimblegen, and Ion Torrent; transcript annotations; small test data for experimenting with pipelines (e.g., for new users).

In some embodiments, data is made available within the context of a database included in a system. Any suitable database structure may be used including relational databases, object-oriented databases, and others. In some embodiments, reference data is stored in a relational database such as a “not-only SQL” (NoSQL) database. In certain embodiments, a graph database is included within systems of the invention. It is also to be understood that the term “database” as used herein is not limited to one single database; rather, multiple databases can be included in a system. For example, a database can include two, three, four, five, six, seven, eight, nine, ten, fifteen, twenty, or more individual databases, including any integer of databases therein, in accordance with embodiments of the invention. For example, one database can contain public reference data, a second database can contain test data from a patient, a third database can contain data from healthy subjects, and a fourth database can contain data from sick subjects with a known condition or disorder. It is to be understood that any other configuration of databases with respect to the data contained therein is also contemplated by the methods described herein.

References and citations to other documents, such as patents, patent applications, patent publications, journals, books, papers, web contents, have been made throughout this disclosure. All such documents are hereby incorporated herein by reference in their entirety for all purposes.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof. All references cited throughout the specification are expressly incorporated by reference herein.

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

While the present invention has been described with reference to the specific embodiments thereof, it should be understood by those skilled in the art that various changes may be made and equivalents may be substituted without departing from the true spirit and scope of the invention. In addition, many modifications may be made to adapt to a particular situation, material, composition of matter, process, process step or steps, to the objective, spirit and scope of the present invention. All such modifications are intended to be within the scope of the claims appended hereto.

### EXAMPLES

### Example 1: Application of Non-Negative Matrix Factorization to TCGA Dataset

To evaluate the application of non-negative matrix factorization for classification of cancer subtypes according to underlying mutational signatures, the TCGA dataset was used.

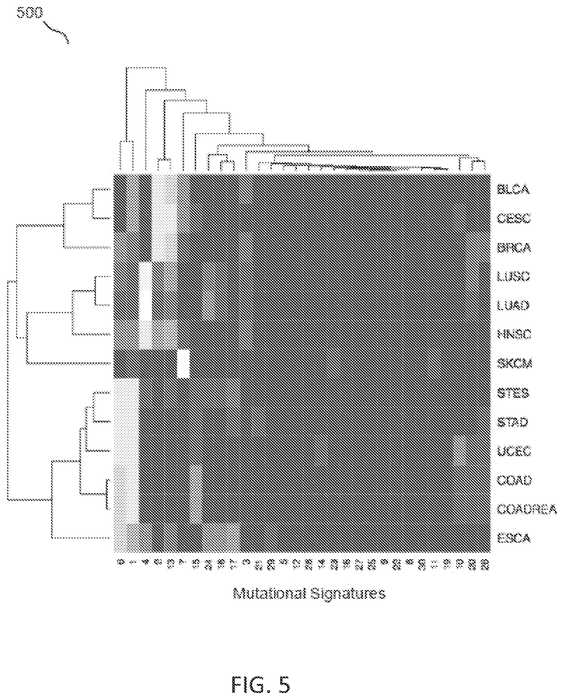

FIG. 5 is a plot 500 showing mutational signatures underlying different cancer types from the TCGA dataset. As shown in plot 500, cancer types (i.e., TCGA cohorts) are represented as rows and mutational signatures are represented as columns. The cohorts are identified using the TCGA identifiers for specific cancer types (acronyms). For example, as known in the art, BRCA is breast cancer, LUSC is lung squamous cell carcinoma, LUAD is lung adenocarcinoma, COAD is colorectal adenocarcinoma, COADREA is a subset of COAD, and HNSC is head and neck carcinoma. As shown in FIG. 5, 30 mutational signatures are clustered across different cancer types. Some of the mutational signatures have been annotated. For example, signature 1 is known to be associated with the spontaneous deamination of 5-methylcytosine, signature 6 is known to be associated with microsatellite instability, and signature 4 is known to be associated with smoking. For each TCGA cohort, the prevalence of patients that have any of the underlying mutational signatures was determined. A high prevalence of a mutational signature within the cohort is represented by white, a moderate prevalence of mutational signatures is represented or yellow and orange coloring and low prevalence of mutational signatures is represented by red. From the clustering profile, one can infer, or determine, cancer types from the underlying mutational signatures. As shown in FIG. 5, signature 1 (spontaneous deamination of 5-methylcytosine) associates with high turnover tissues, e.g., COAD and COADREA; signature 6 (defective DNA mismatch repair and microsatellite instability) associates with colorectal cancer (COAD); and signature 4 (smoking) associates with HNSC, LUSC, and LUAD.

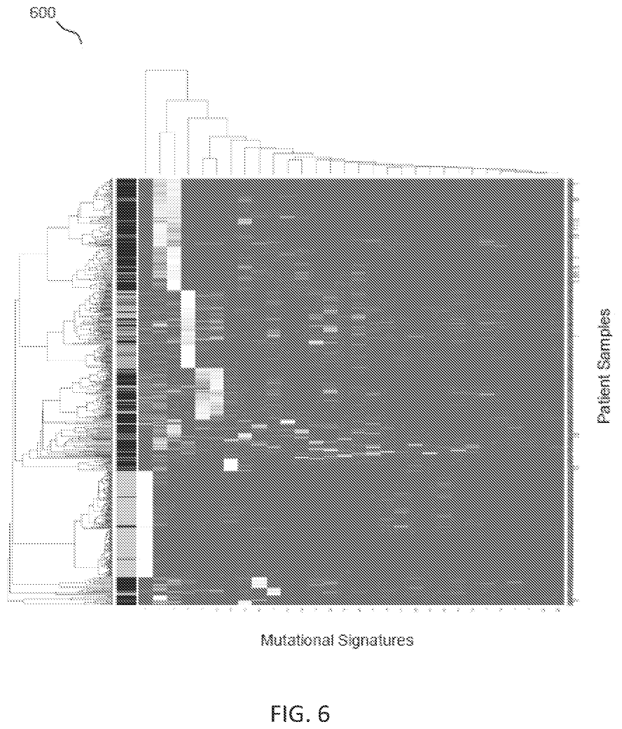

In accordance with the present invention, non-negative linear regression was applied to each individual TCGA patient sample of FIG. 5. FIG. 6 is a plot 600 showing a hierarchical clustering of individual TCGA patient samples according to identified mutational samples. In plot 600, TCGA patient samples are represented as rows and mutational signatures are represented as columns. Each TCGA patient sample is clustered according to the mutational signatures.

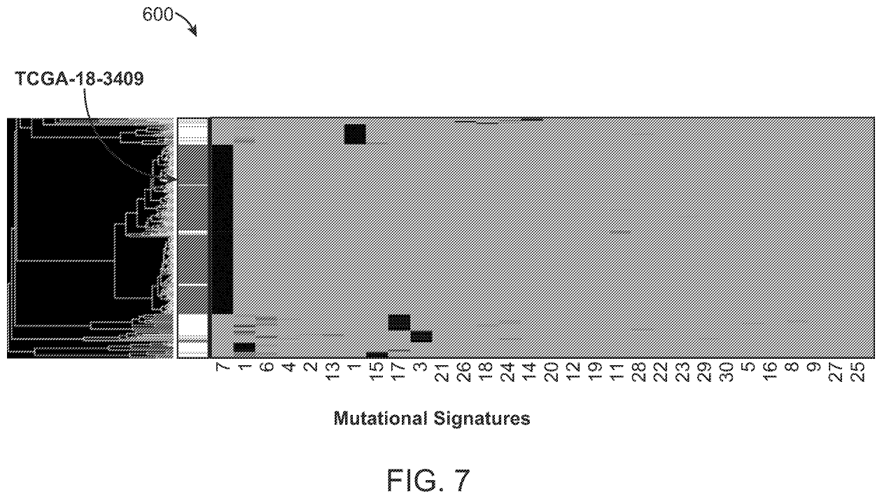

FIG. 7 is an enlarged view of a portion of plot 600 of FIG. 6 showing clustering of a lung squamous cell carcinoma patient sample (identified on FIG. 7 as TCGA-18-3409) within a cluster of known melanoma patient samples. The mutational signatures associated with the TCGA-18-3409 sample suggest that the cancer type is more closely related to skin cancers than to lung cancers.

The clinical notes for the TCGA-18-3409 patient (not shown) indicate that the TCGA-18-3409 patient has a prior malignancy of basal cell carcinoma (a non-melanoma). An analysis (data not shown) of the individual genes that are affected in the TCGA-18-3409 patient sample shows that the PTCHD1, 2, and 4 genes all include missense mutations. PTCHD1 is suspected to have a similar inhibitory function to PTCH1, a gene that is commonly mutated in basal cell carcinomas. Reported estimates of malignant basal cell carcinoma vary widely, ranging from about 0.0028% to about 0.55% of all basal cell carcinomas, with about 28% of sites having metastases to lung and about 11% to skin/soft tissue. This is in line with what is observed in the TCGA-18-3409 patient sample and reported in the clinical notes. This example demonstrates that classifying patients based on the mutational signatures alone may provide a more robust identification of the type of cancer that a patient has as opposed to just reporting the location of where a malignancy is detected and excised.

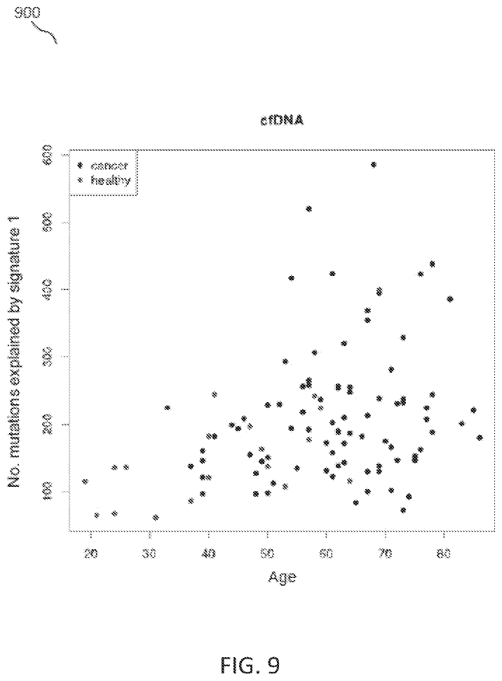

Aspects of the invention include identifying mutational signatures in healthy patients and utilizing the mutational signatures in the detection, diagnosis and/or classification of cancer. For example, FIG. 9 is a plot 900 showing the estimated number of signature 1 mutations identified in cfDNA samples from both cancer patients and healthy subjects as a function of age. As shown in FIG. 9, there is a strong correlation of signature 1 mutations with age in healthy subjects (red dots). The strong correlation of signature 1 mutations with age suggests that signature 1 can be used to inherently account for the aging process in variant calling in a cfDNA sample.

Also, as shown in FIG. 9, there is a strong correlation of signature 1 mutations with age in cancer patients (black dots) and healthy subjects (red dots). Although not wishing to be bound by theory, it is believed that if a signature 1 contribution in a subject diverges significantly from the characteristic signature 1 contribution with age for healthy subjects, that there is either an acceleration or slowdown in cell cycle turnover. Accordingly, in some embodiments, the divergence, or variance, between a test patient's signature 1 profile and a characteristic signature 1 profile determined for healthy subjects at a given age can be used as a classification signature to distinguish healthy and diseased subjects from one another (i.e., the signature 1 contribution could itself be a test for cancer).

### Example 2: Identification of Cancer from a Mutational Signature Observed in a New Patient Sample

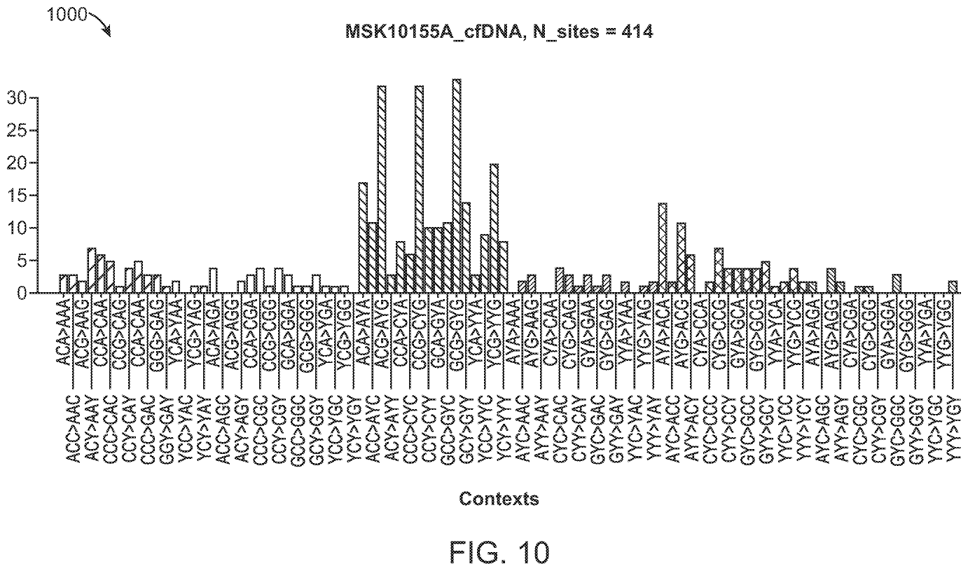

FIG. 10 is a bar graph 1000 showing an example of a mutational profile from a patient's cfDNA sample (MSK10155A). The mutational profile was constructed based on the triplet sequence context of base substitution mutations in the patient's cfDNA as described with reference to FIG. 2.

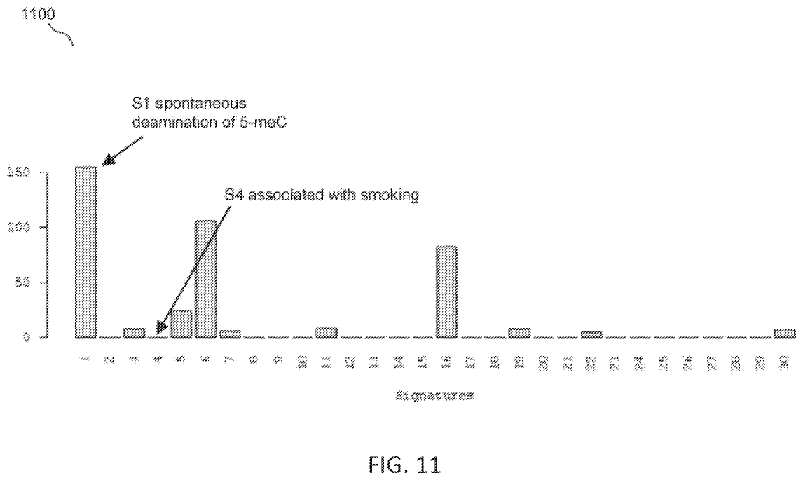

FIG. 11 is a bar graph 1100 showing the number of observed base substitution mutations of FIG. 10 for each underlying mutational signature context. The mutational signature shown in plot 1000 is a combination of the 30 underlying mutational signatures that account for the patient's cfDNA mutational profile. Each bar on the graph represents an underlying mutational signature. For example, the fourth bar on the graph represents signature 4, which is associated with mutations induced by smoking. A prediction based on the relatively low number of mutation counts mapping to signature 4 would be that this patient has no smoking history. The first bar on the graph represents signature 1, which is associated with the spontaneous deamination of 5-methylcytosine and is a contribution from the number of cell cycle turnovers. In tumor tissue biopsy sequencing, it has been reported that the signature 1 process is a clock-like mutational process that occurs in human somatic cells over time.

### Example 3: Detection of APOBEC Signature

The APOBEC mutational signature was detected in cfDNA from a breast cancer patient (patient sample MSK11591A). Patient sample MSK11591A is different from other cohort patient samples by multiple features.

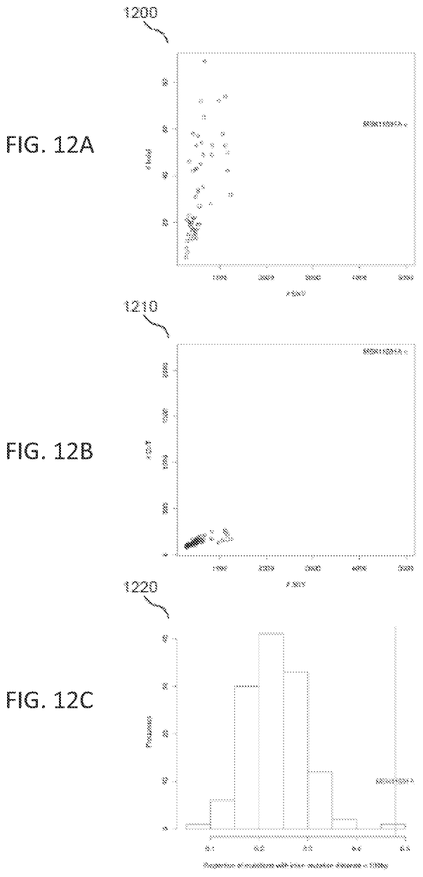

FIG. 12A is a plot 1200 showing the SNV and indel burden in cfDNA from sample MSK11591A. The data show a high number of point mutations (SNVs) and indels in sample MSK11591A.

FIG. 12B is a plot 1210 showing the number of C>T base substitutions in sample MSK11591A. The data show that point mutations (SNVs) in sample MSK11591A are largely C>T mutations.

FIG. 12C is a bar graph 1220 showing the distribution of mutations with inter-mutation distance <100 bp in sample MSK11591A and other cohort cfDNA patient samples. For each sample, the inter-mutation distance (i.e., the distance from any given mutation to the next closest somatic mutation), was calculated. In sample MSK11591A, about 50% of mutations are within about 100 bases of each other compared to the distribution of inter-mutation distance for mutations in other cfDNA patient samples. The data show that mutations in sample MSK11591A are highly clustered.

The high mutation burden in sample MSK11591A is derived from biological signals and is not a contribution of technical artifacts (e.g., sample passed quality control metrics; data not shown).

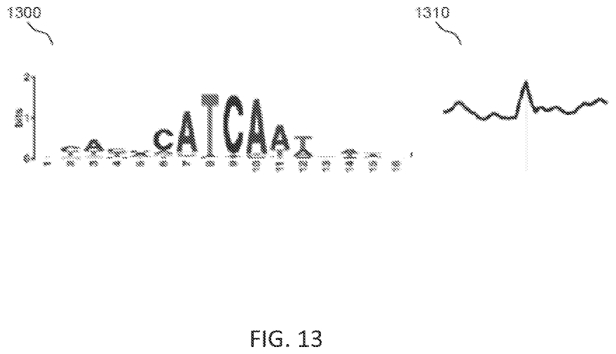

A motif detection approach was used to identify enrichment of sequence context around each mutation in sample MSK11591A by identifying sequences shared between the regions surrounding somatic mutations in MSK11591A that occur more frequently than is expected by chance. FIG. 13 shows a plot 1300 of sequence context and a plot 1310 of motif location relative to SNVs in sample MSK11591A. Referring to plot 1300, the mutations are enriched for TCA sequence motifs. The height of each base (ATCG) in plot 1300 represents the information content of the motif. Referring to plot 1310, the TCA motif is centrally localized relative the SNVs in sample MSK11591A.

Mutations in sample MSK11591A are primarily C>T mutations that are clustered and enriched for TCA sequence motifs. A possible explanation for this mutation pattern in sample MSK11591A is APOBEC-mediated hypermutation. APOBEC (apolipoprotein B mRNA editing enzyme, catalytic polypeptide-like) is involved in innate immunity against viral infections and in RNA editing, usually outside of the nucleus. APOBEC is a family of single stranded DNA-specific cytidine deaminases. APOBEC deaminates cytosine preferentially at the TCW motif (W=A or T) and introduces C>T and C>G substitutions. APOBEC activity has a systematic strand bias and induces spatial clustering of mutations. The APOBEC mutation pattern (TCW mutation context; W=A or T) has been shown to occur in multiple cancer types (e.g., breast cancer, lung cancer, and head and neck cancer).

From the analysis of the cfDNA sample MSK11591A, it is likely that the patient has an ABOPEC-driven process as an underlying contribution to mutations. In sample MSK11591A cfDNA, the APOBEC signature is detected and this signature can be traced back to the non-negative matrix factorization analysis, where it is referred to as signature 2 in the matrix assignment.

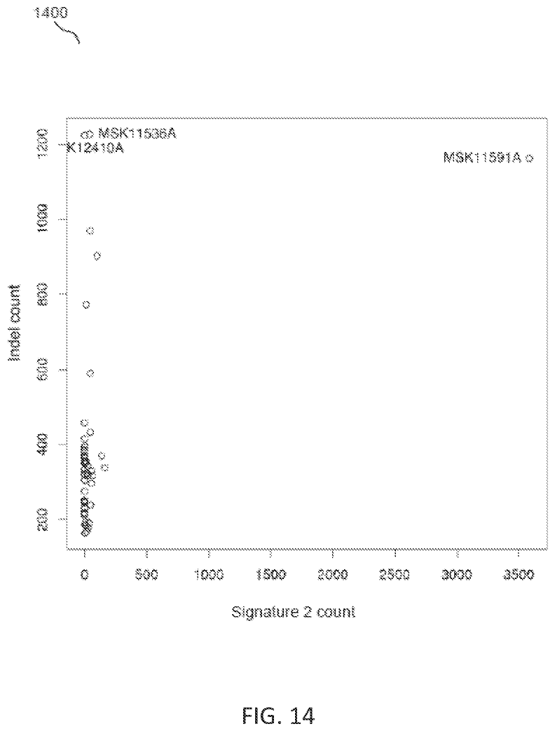

FIG. 14 is a plot 1400 showing the inferred signature 2 (APOBEC) point mutation count versus indel count in cfDNA samples with MSK11591A labelled. Sample MSK11591A distinguished from the remaining samples by a high signature 2 exposure and indel exposure, improved stratification relative to FIG. 12A.

About 80% of mutations in sample MSK11591A can be attributed to the APOBEC signature 2. Analysis of sequencing data from a peripheral blood mononuclear cell (PBMC) sample from the MSK11591A patient shows that about 9% of the variants identified in cfDNA are also found in PBMCs (data not shown), which suggests an APOBEC mutation arose early during development in this patient.

Other biological features associated with the APOBEC mutational signature 2 can be combined with the mutational signature data in order to refine assignments/classification of a patient sample. For example, the APOBEC signature 2 may be associated with overexpression (e.g., amplification) of HER2 in breast cancer patients.

From the analysis of sample MSK11591A cfDNA, it is predicted that the patient has kataegis. Kataegis is a mutational process observed in cancer that results in hypermutation in localized genomic regions. A high mutation burden and clustering of mutations in sample MSK11591A cfDNA were described with reference to FIGS. 12A, 12B, and 12C.

Hypermutation can generate a high neoepitope load within a patient. Neoepitopes are targets for immunotherapy. Identification of the APOBEC mutational signature in cfDNA from a patient sample can be used to classify patients for different types of therapies (e.g., immunotherapy).

### Example 4: Monitoring Mutational Signatures at Multiple Time Points

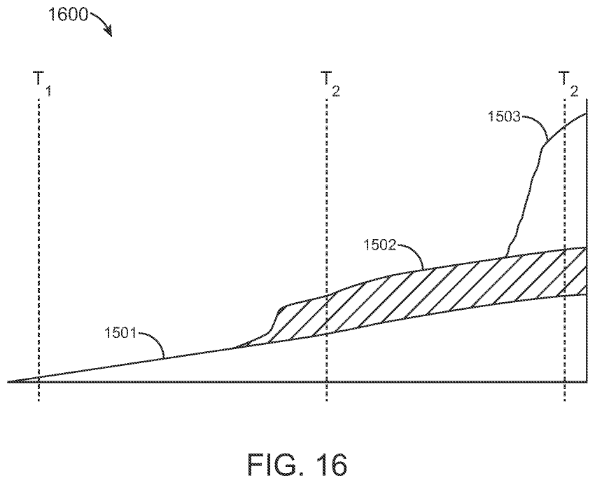

The change in mutational signature proportions in an individual through time can be monitored for detection of cancer, monitoring cancer progression, and/or monitoring of cancer treatment. FIG. 16 represents a simulation showing the monitoring of three mutational signatures over time, spontaneous deamination 1501 (COSMIC signature 1); cigarette smoke exposure 1502 (COSMIC signature 4); and AID/APOBEC hypermutation 1503 (COSMIC signature 2). Mutations accumulate within the individual over time as a function of endogenous and exogenous mutational processes. As a result, the cumulative number of mutations is monotonically increasing over time. This is shown in FIG. 16, where the width of each band represents the cumulative mutational load, or mutational signature load, in that individual through time.

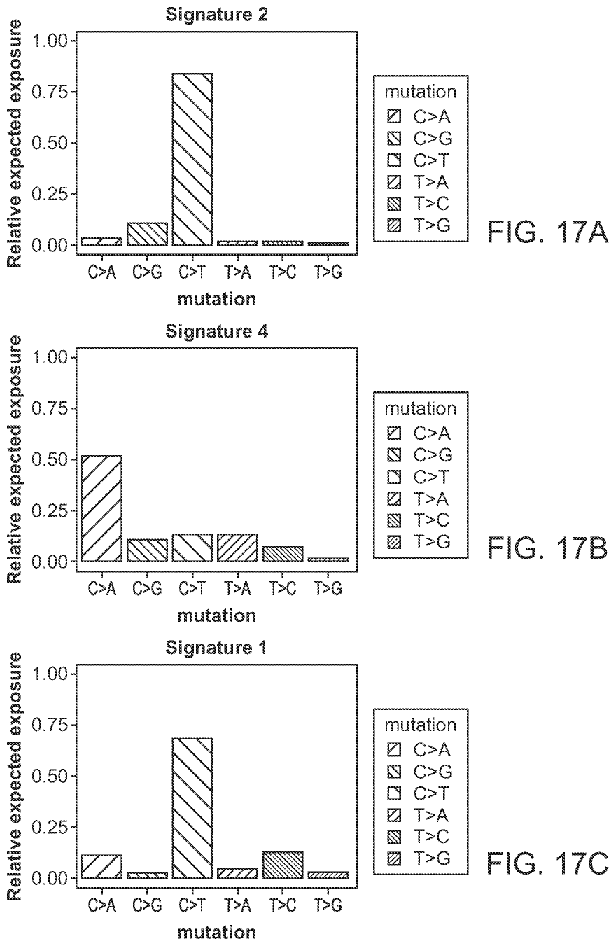

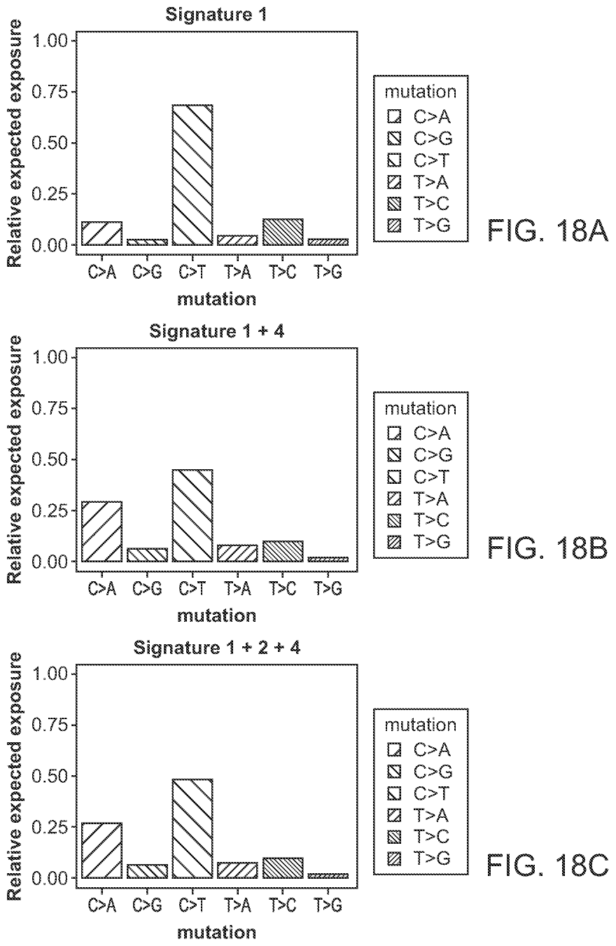

Mutations or mutation profiles (as shown in FIGS. 18A, B and C) can be identified, and changes therein monitored through time, by obtaining test samples from a patient at multiple time points. For example, as shown in FIG. 16, test samples may be obtained from a patient at a first time point (T₁), a second time point (T₂), and a third time point (T₃) (shown as dotted vertical lines), and nucleic acids obtained therefrom sequenced and used to call mutations or variants at each time point. For each time point a mutation count histogram from the superposition of mutational signatures can be determined (shown in FIGS. 18A, B and C). These mutational count histograms may be a combination of expected histograms (shown in FIGS. 17A, B and C) (FIGS. 17A-C show mutational count histograms determined from the aggregation of 96 trinucleotide mutational contexts to the six single base change contexts for: (A) AID/APOBEC hypermutation; (B) cigarette smoke exposure; and (C) spontaneous deamination). For example, as shown, the mutational count histogram at time point T₂ (FIG. 18B) is a combination of the mutational signatures expected for spontaneous deamination (FIG. 17C) and cigarette smoke exposure (FIG. 17B). Likewise, as shown, the mutational count histogram at time point T₃ (FIG. 18C) is a combination of the mutational signatures expected for spontaneous deamination (FIG. 17C), cigarette smoke exposure (FIG. 17B) and AID/APOBEC hypermutation (FIG. 17A).

As shown in FIG. 16 spontaneous deamination 1501 occurs at a rate proportional to the number of cell divisions. At the onset of increased proliferation in a tumor the cumulative amount of mutations from spontaneous deamination 1501 is increased following an increased rate of cell division. The increase in spontaneous deamination is potentially a distinguishing feature of cell cycle dysregulation that can differentiate individuals with cancer from individuals without cancer. Dysregulation would be detected as follows: given a model of the spontaneous deamination mutation process as a function of time identify increased rate in cell division rate in cell-free nucleic acids (e.g., cfDNA) by assessing deviation from expectation conditional on the individual's reported age, ethnicity, genetic background, white-blood cell somatic variants, gender, known mutational exposures, and clinical history.

At time point T₃, the AID/APOBEC hypermutation 1503 process can be detected, and may be indicative of the development of cancer. In a patient with cancer, the AID/APOBEC hypermutation 1503 signature would be expected to show greater intensity than the cigarette smoke exposure 1502 signature per unit time. Increased intensity detected at T₃ reflect hypermutation within a cell and/or increased proliferation. Comparison the velocity of spontaneous deamination mutational process 1501 at T₃ to that determined at earlier time points T₁ and T₂ indicates that cell proliferation has not increased (as the spontaneous deamination mutational signature at T₃ is proportional to cell division rate). Accordingly, we can conclude that hypermutation is the underlying cause of the increased mutation rate observed at T₃.

Cigarette smoke exposure 1502 (mutational signature 4) is an environmental exposure and increases in proportion with exposure to cigarette smoking in an individual. In this simulation the individual stops smoking and as a result mutations induced by smoking do not increase from time point T₂ to T₃.

### Example 5: Supervised Mutational Signature Deconvolution

Supervised mutational signature deconvolution involves determining a projection of a mutational profile onto a basis of mutational signatures, such as, without limitation, known mutational signatures 1-30 described on the COSMIC website (referenced above). Since mutational processes are either active or inactive, and only a subset of mutational processes are active in any individual patient, analysis involves determining whether the estimated exposures have non-negative values. Additionally, since mutational signatures can share sequence contexts, analysis also involves “regularizing” the coefficient estimates to shrink estimates towards zero. In other words, the analyses described herein seek to perform variable selection and shrinkage to isolate the important mutational processes out of the set of specified mutational signatures. Two techniques known for this include ridge regression and the lasso. In this example, elastic net non-negative least squares regression is used (Mandal & Ma, Computational Statistics and Data Analysis, 2016, the disclosure of which is hereby incorporated by reference herein). In statistics, and in particular, in the fitting of linear or logistic regression models, the elastic net is a regularized regression method that linearly combines the L1 and L2 penalties of the lasso and ridge methods. Further details are provided, for example, in Zou, Hui, and Trevor Hastie, “Regularization and variable selection via the elastic net.” Journal of the Royal Statistical Society: Series B (Statistical Methodology) 67.2 (2005): 301-320, the disclosure of which is incorporated herein by reference in its entirety.

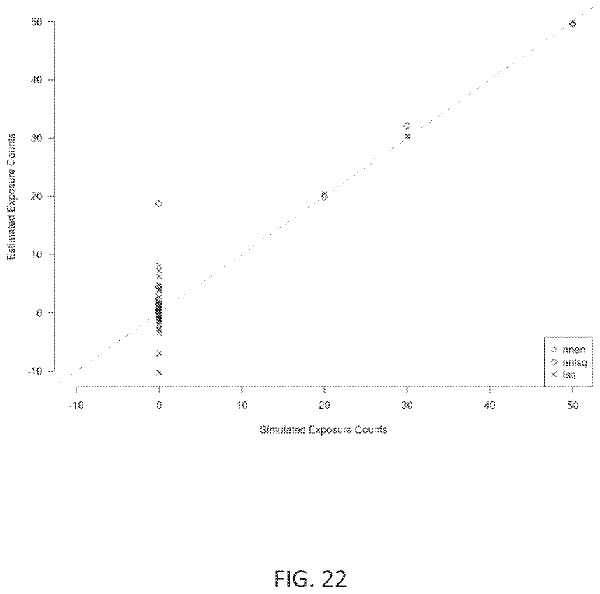

In FIG. 22, an example of different regression approaches applied to a simulated mutational profile is provided. In the simulation, an individual subject has 100 mutations that manifest from a combination of 0.3 (30%)×Signature 1; 0.5 (50%)×Signature 2; and 0.2 (20%)×Signature 13, with some uniform noise across the 96 trinucleotide context single nucleotide mutations. The consequence of applying least squares linear (lsq) regression is that fit negative coefficients (exposures) are estimated for some signatures. Non-negative least squares regression (nnlsq) eliminates negative coefficients, but can lead to overestimation of total mutational burden and spurious non-zero coefficients. Elastic net non-negative least squares regression (nnen), guards against both of these properties.

The results provided in FIG. 22 demonstrate that regression analysis can be successfully used to demonstrate that regression analysis can be successfully used to determine the exposure weight, or percentage, of each mutational signature within a sample (i.e., deconvolution of a mutational profile into a combination of mutational signatures). The subject methods therefore facilitate determination of the relative contribution of each mutational signature to a patient's mutation profile, thereby facilitating identification of the type of mutational processes that are operative within the patient, as well as quantifying the relative contribution of each mutational process.

### Example 6: Comparison of Sequence Context of WBC and cfDNA

Different tissue types have different somatic variant profiles, and white blood cell (WBC) somatic variants can be used as a basis for comparison to other tissues. In this example, three different subjects were evaluated to determine the somatic variant content of different tissues, and the relative levels of cfDNA somatic variants and WBC somatic variants were compared. The first subject was a 72 year old human patient with colorectal cancer and microsatellite instability (MSI) (“the MSI patient”). The second subject was an 85 year old human patient who did not have cancer (“the 85 year old patient”), and the third subject was a 68 year old human patient who did not have cancer (“the 68 year old patient”).

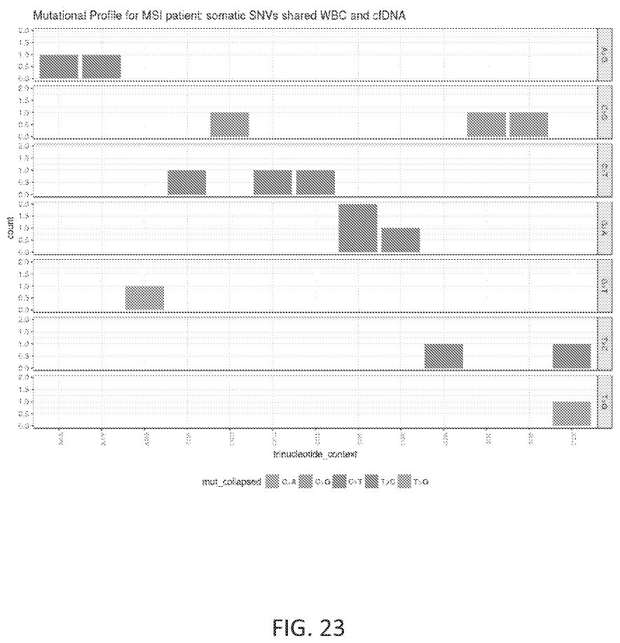

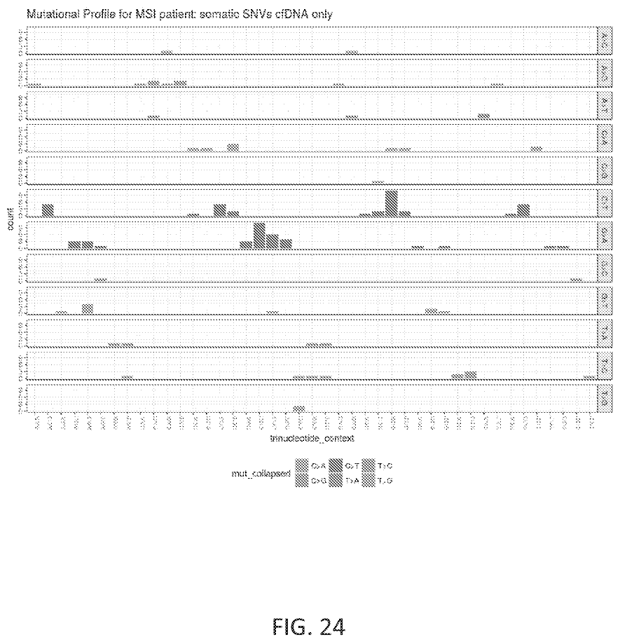

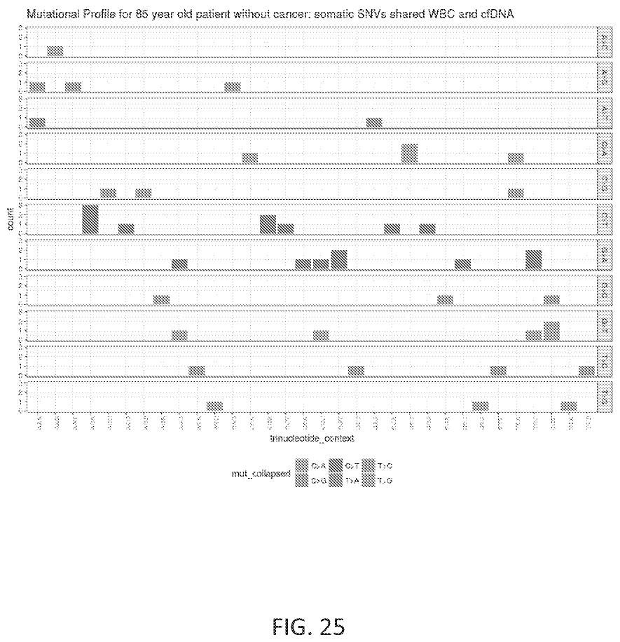

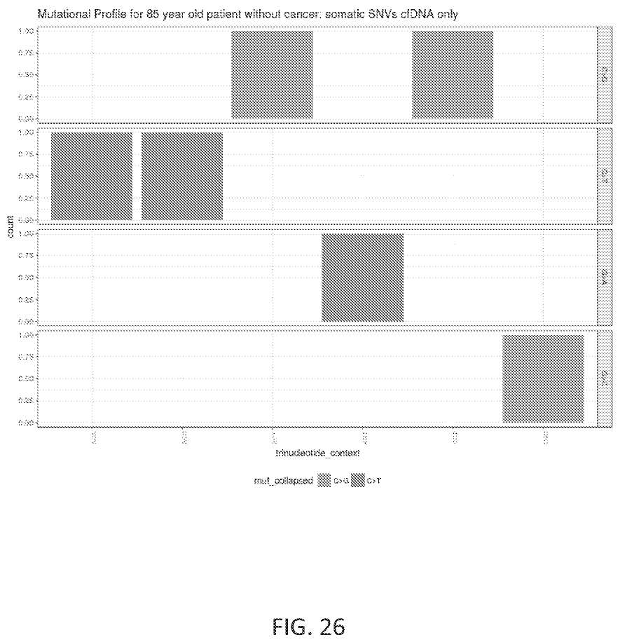

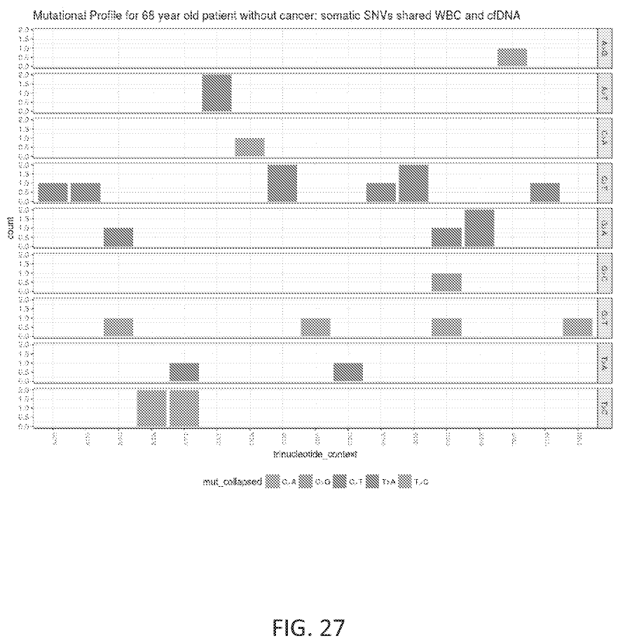

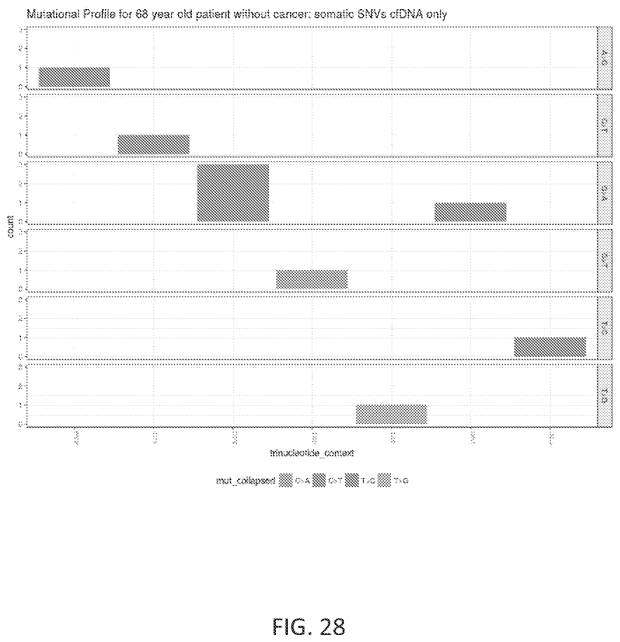

FIG. 23 shows the trinucleotide context of mutations represented on the x-axis and the number of mutations on the y-axis for WBC and cfDNA SNVs for the MSI patient. FIG. 24 shows the same data, but only for the cfDNA SNVs (WBC SNVs removed). Mutations are presented relative to the reference sequence context of GRCh37 (there are 64 different trinucleotide contexts after accounting for reverse complementarity; mutations were not reverse complemented). This comparison reveals that the MSI patient has more cfDNA SNVs that are not common to, or shared by, the WBC SNVs. The data for the 85 year old patient and the 68 year old patient, presented in FIGS. 25, 26, 27 and 28, demonstrate that non-cancerous patients have a lower number of SNVs after accounting for WBC SNVs.

### Example 7: Molecular Classification of Patient Samples

The subject methods facilitate determination of specific mutational processes that are active within an individual, thereby allowing molecular classification of disease, and selection of appropriate treatment based on the molecular classification, which can be used in place of or in conjunction with other metrics, such as, e.g., tumor location, tissue type, etc. Importantly, the subject methods can facilitate identification of an active mutational process within a patient before traditionally observable clinical symptoms arise. Furthermore, the subject methods are valuable even if clinical symptoms are present, as is the case with, e.g., checkpoint inhibitor therapy, which is currently administered to individuals with MSI, who are typically late-stage patients.

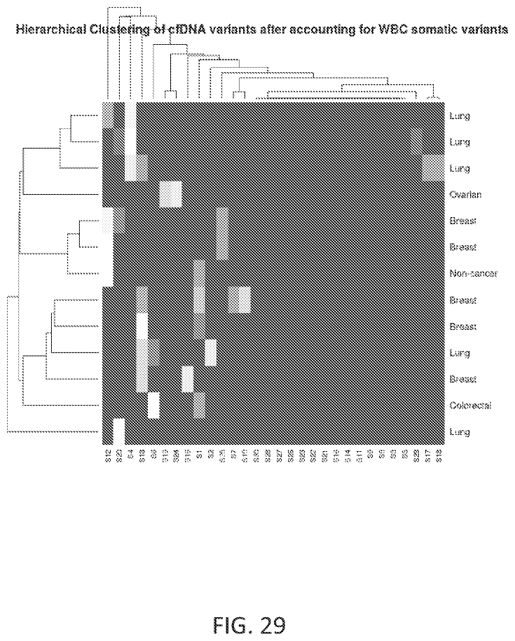

FIG. 29 is a “heat map” showing 30 different known mutational signatures along the x-axis, and showing the relative abundance of each signature in each individual, including cancers from different tissues, and provides a hierarchical clustering across inferred mutational signature exposures for cfDNA test samples using Euclidean distance. FIG. 29 includes data from one individual who self-identified as healthy, and is therefore labeled as “non-cancer”. However, this individual has an extremely high SNV load, which indicates that disease may be present, even though observable clinical symptoms have not yet surfaced.

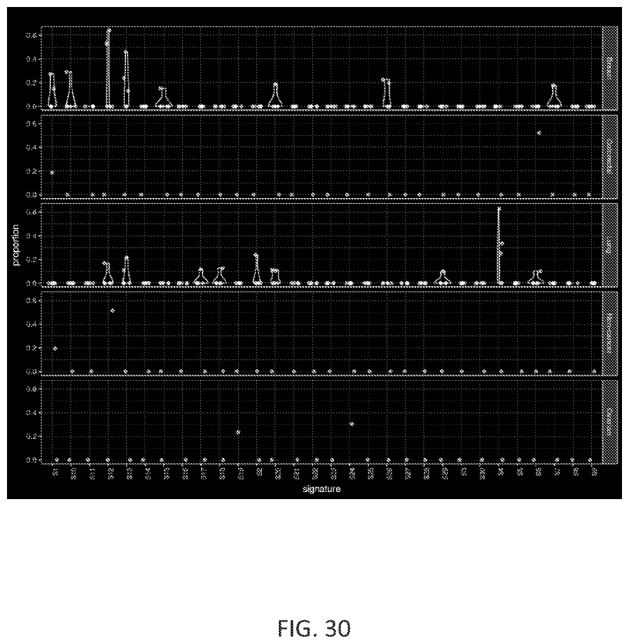

Global behaviors for some signatures associated with environmental exposures were also observed. For example, Signature 4, which is associated with exposure to cigarette smoke, is clearly observed in Lung cancer samples (FIG. 30). This demonstrates that different mutational processes are active within different samples, and provides a molecular classification for different cancers. For example, a patient who shows high activity of Signature 4 (smoking) could benefit from treatment approaches that are targeted toward this mutational process. Notably, the healthy individual included in this analysis shows high activity of Signature 12, indicating that this individual may be in the early stages of disease, before clinical symptoms have surfaced. The subject methods facilitate identification of such individuals at the early stages of disease, when therapeutic intervention has a greater chance of success.

To account for different amounts of certainty in estimating signature exposures for each signature, an evidence threshold for each contributing signature was applied. For example, Signature 3 has a broad probability distribution across almost all 96 trinucleotide contexts, and is therefore vulnerable to having the magnitude of its coefficient overestimated. In addition, evidence thresholds for signatures associated with high mutational load, like Signature 7 (UV exposure) and Signature 10 (defective POLE), can be applied to match the expected biology of those signatures. Signatures with an exposure proportion less than 0.1 (on a scale from zero to one) can be set to an exposure proportion of zero. In this example, Signatures 3, 7, and 10, which had less than 30 supporting mutations, were set to an exposure proportion of zero.

### Example 8: Detection of Mutational Signatures in Combination with Fragment Length Profiling

Signature 12 has only been observed in liver cancer in COSMIC analyses. Signature 12 exhibits a strong transcriptional strand bias for T>C substitutions. In this example, exposure to signature 12 was observed in a subject who self-reported as healthy (i.e., not having cancer) and in subjects with cancer other than liver cancer. To assess whether these observed variants were likely derived from solid tissue, or potentially tumor, the median fragment lengths for reads supporting the mutant allele were compared to the reference allele at mutants candidates. All samples showed a length shift to shorter fragments, increasing the confidence that the observed SNVs were due to a mutational process, and not derived from a sequencing artifact. Use of fragment length profiling of cfDNA samples is known in the art, and includes, for example, the techniques described in US Patent Application Publication Nos. 2013/0237431 and 2016/0201142, the disclosures of which are incorporated by reference herein in their entirety.

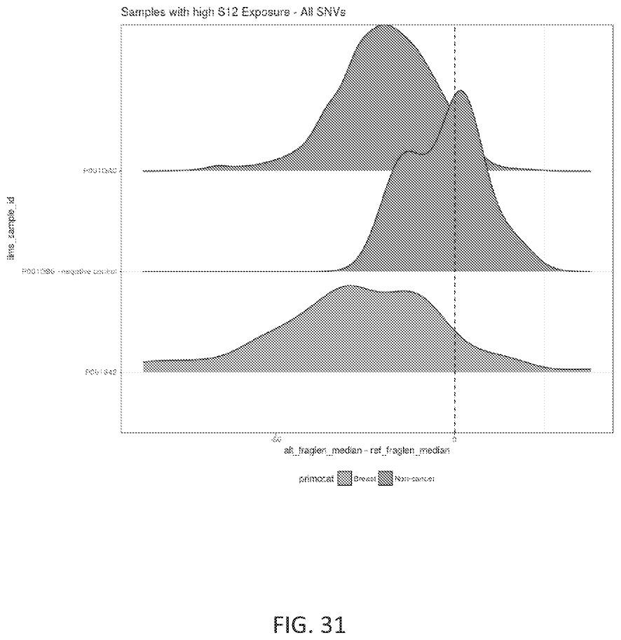

FIG. 31 shows cfDNA fragment length data across all SNVs obtained from subjects with high Signature 12 exposure. The lower-most distribution was obtained from a subject with breast cancer, and shows that the fragment length distribution is shifted to the left, away from the vertical dashed line (which indicates the location where the peak of the fragment length distribution is anticipated to occur in healthy control samples). The upper-most distribution was obtained from a subject who self-reported as healthy, but whose analysis revealed a high level of exposure to Signature 12. In agreement with the Signature 12 exposure observation, the fragment length distribution for this subject was shifted to the left, which indicates shorter cfDNA fragment lengths, and possible presence of cancer. The middle distribution is from a negative control sample (i.e., a non-cancer sample), and shows that the fragment length distribution aligns with the vertical dashed line, as anticipated.

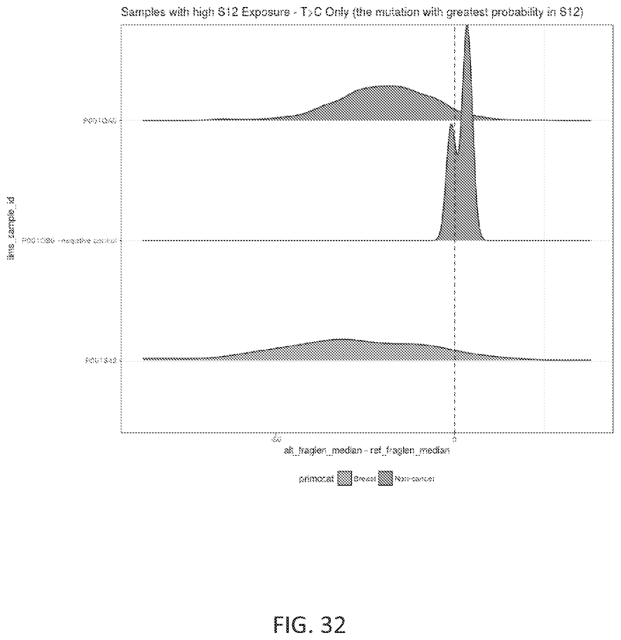

FIG. 32 shows the same analysis, but with T>C mutations only. This is the mutation with the greatest probability in Signature 12. When the T>C mutations are analyzed separately from all of the SNVs, the differences in the fragment length distribution profiles are more pronounced, and clearly show a shift toward shorter fragment lengths from the samples that contain high Signature 12 exposure. These data demonstrate that fragment length profiling can be used in conjunction with the subject methods to provide further confidence in the detection of active mutational processes.

### Example 9: Detection of Smoking-Associated Signature 4

Signature 4 is associated with tobacco smoking (and tobacco smoking carcinogens such as benzo[a]pyrene). It has been found in head and neck cancer, liver cancer, lung adenocarcinoma, lung squamous cell carcinoma, small cell lung carcinoma, and esophageal cancer. Signature 4 exhibits a transcriptional strand bias for C>A mutations, compatible with the notion that damage to guanine is repaired by transcription coupled nucleotide excision repair. Signature 4 is also associated with CC>AA substitutions. More information relating to Signature 4 (and other signatures) can be found online at the Catalog of Somatic Mutations In Cancer (COSMIC) website, at http://cancer.sanger.ac.uk/cosmic/signatures.

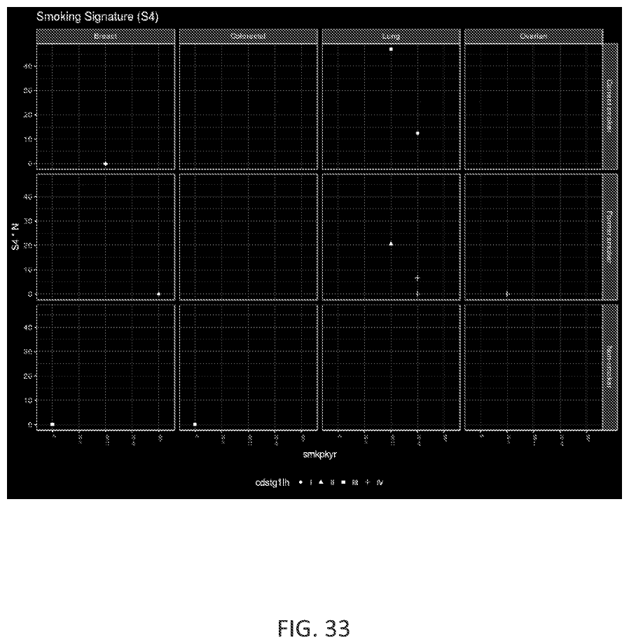

FIG. 33 shows Signature 4 exposure levels across individuals, plotted as a function of smoking exposure and smoking history. The pack-year (x-axis label) is a unit for measuring the amount a person has smoked over a long period of time. It is calculated by multiplying the number of packs of cigarettes smoked per day by the number of years the person has smoked. For example, 1 pack-year is equal to smoking 20 cigarettes (1 pack) per day for 1 year, or 40 cigarettes per day for half a year. This figures indicates that individuals with lung cancer who have a current or prior smoking history have Signature 4 exposure.

The data in FIG. 33 show that, as anticipated, subjects who are current or former smokers have high Signature 4 exposure. This is demonstrated across multiple cancer types. These data demonstrate that clinical data (such as patient-reported smoking history) can be used in conjunction with the subject methods to provide further confidence in the detection of active mutational processes.

### Example 10: Detection of Defective DNA Mismatch Repair-Associated Signature 6

Signature 6 has been found in 17 cancer types and is most common in colorectal and uterine cancers. In most other cancer types, Signature 6 is found in less than 13% of examined samples. Signature 6 is associated with high numbers of small (shorter than 3 base pairs) insertions and deletions at mono- or polynucleotide repeats. Signature 6 is one of 4 mutational signatures associated with defective DNA mismatch repair, and is often found to co-occur with Signatures 15, 20, and 26. Microsatellite instability (MSI) tumors in 15% of sporadic colorectal cancer result from the hyper-methylation of the MLH1 gene promoter, whereas MSI tumors in Lynch syndrome are caused by germline mutations in MLH1, MSH2, MSH6, and PMS2. More information relating to Signature 6 (and other signatures) can be found online at the Catalog of Somatic Mutations In Cancer (COSMIC) website, at http://cancer.sanger.ac.uk/cosmic/signatures.

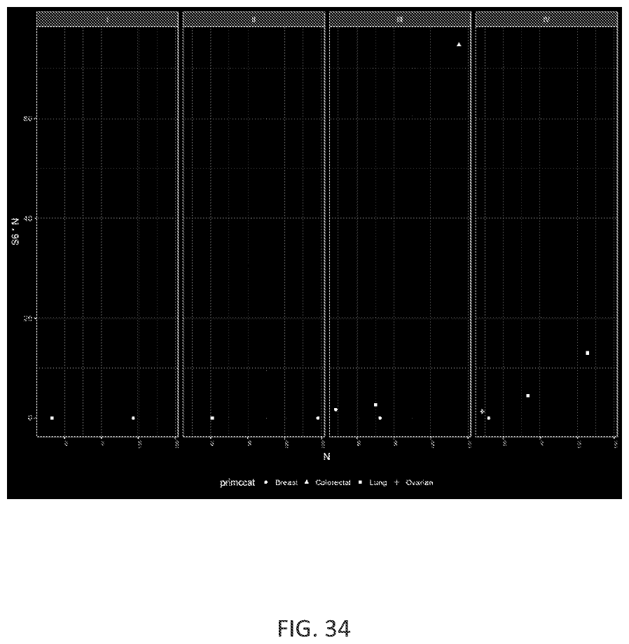

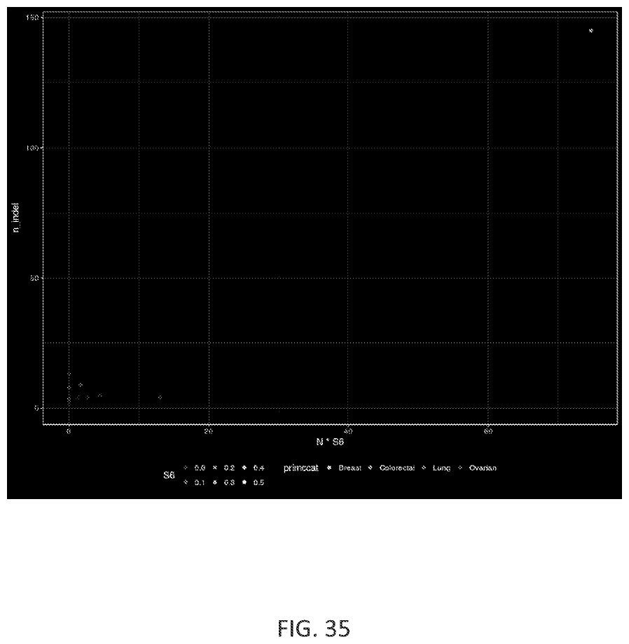

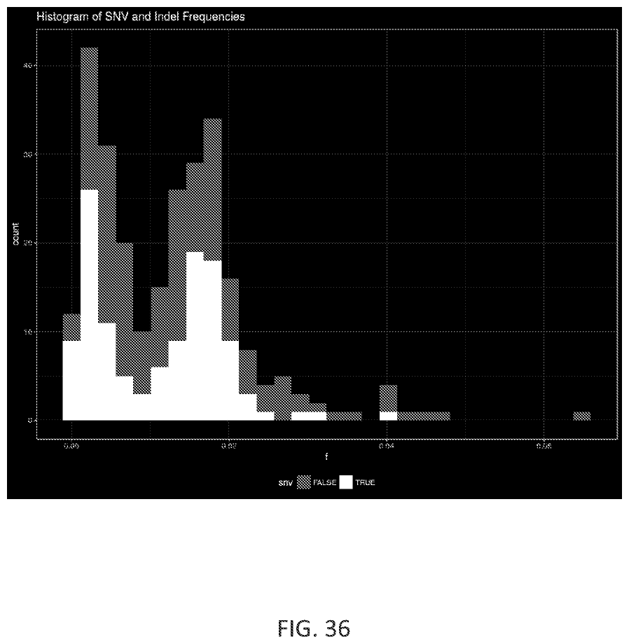

FIG. 34 shows Signature 6 exposure plotted across different cancer types. As anticipated, high exposure levels to Signature 6 (>60%) was observed in a colorectal cancer sample. The association of Signature 6 exposure with high numbers of indels is demonstrated in FIG. 35, which shows the number of observed indels (y-axis) v. Signature 6 exposure in absolute SNV count (x-axis). FIG. 36 shows a histogram of SNV and indel frequencies (ALT reads/(ALT reads+REF reads)), which is compatible with the same generative process for SNVs and indels. This observation increases the confidence that the observed level of Signature 6 exposure is correct, due to the known association between Signature 6 and increased indels. The shared sequence context of indels (Table 1) is compatible with microsatellite instability and supports a mutational signature of defective DNA mismatch repair. Table 1, below, shows the data corresponding to the reference allele, the alternative allele, and the number of occurrences.

TABLE 1

| Reference_ | Alternative_ | Number of |
|---|---|---|
| allele | allele | Occurrences |
| A | AC | 1 |
| A | AG | 1 |
| AAAAG | A | 1 |
| AAAG | A | 1 |
| AAG | A | 1 |
| AC | A | 5 |
| ACCCCACCCC | A | 1 |
| AGCC | A | 1 |
| AT | A | 8 |
| ATT | A | 2 |
| ATTT | A | 3 |
| ATTTTT | C | 2 |
| CA | C | 2 |
| CAAA | C | 2 |
| CAAAAA | C | 1 |
| CAAAAG | C | 1 |
| CAAG | C | 1 |
| CAGAG | C | 1 |
| CCTGCTG | C | 1 |
| CG | C | 1 |
| CT | C | 3 |
| CTCCCTTCCTCACTGGGATTCAGAG | C | 1 |
| CTT | C | 7 |
| CTTT | C | 3 |
| CTTTT | C | 3 |
| CTTTTT | C | 5 |
| CTTTTTT | C | 3 |
| CTTTTTTT | C | 3 |
| CTTTTTTTT | C | 3 |
| CTTTTTTTTT | C | 1 |
| CTTTTTTTTTTT | C | 1 |
| CTTTTTTTTTTTTT | C | 1 |
| CTTTTTTTTTTTTTT | C | 1 |
| G | GA | 1 |
| G | GGTT | 1 |
| G | GT | 1 |
| GA | G | 8 |
| GAA | G | 2 |
| GAAA | G | 3 |
| GAAAA | G | 2 |
| GAAAAA | G | 1 |
| GC | G | 2 |
| GT | G | 2 |
| GTC | G | 1 |
| GTTT | G | 1 |
| GTTTT | G | 1 |
| GTTTTT | G | 2 |
| GTTTTTT | G | 1 |
| GTTTTTTT | G | 1 |
| GTTTTTTTT | G | 1 |
| GTTTTTTTTT | G | 1 |
| T | TA | 1 |
| T | TATG | 1 |
| T | TC | 2 |
| T | TG | 2 |
| TA | T | 10 |
| TAA | T | 2 |
| TAAA | T | 3 |
| TAAAA | T | 4 |
| TAAAAA | T | 3 |
| TAAAAAA | T | 2 |
| TAAAAAAA | T | 2 |
| TAAAAAAAA | T | 1 |
| TAC | T | 1 |
| TAGCAGC | T | 1 |
| TC | T | 5 |
| TCC | T | 1 |

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

1. A computer-implemented method for detecting the presence of a cancer in a patient, the method comprising:
receiving a data set in a computer comprising a processor and a computer-readable medium, wherein the data set comprises a plurality of sequence reads obtained by sequencing a plurality of nucleic acids in a biological test sample from the patient, and wherein the computer-readable medium comprises instructions that, when executed by the processor, cause the computer to:
identify one or more somatic mutations in the biological test sample;
generate a somatic mutational profile that comprises the one or more somatic mutations;
deconvolute the somatic mutational profile into one or more mutational signatures; and
determine one or more exposure weights for one or more of the mutational signatures; and
detecting the presence of the cancer in the patient based on the one or more exposure weights of the one or more mutational signatures.

2. The method of claim 1, wherein the one or more somatic mutations are identified by aligning the plurality of sequence reads to a reference genome.

3. The method of claim 1, wherein the one or more somatic mutations are identified by performing a de novo assembly procedure on a plurality of sequence reads.

4. The method of claim 1, wherein the presence of cancer in the patient is detected from the one or more exposure weights of the one or more mutational signatures using a supervised approach, wherein the one or more exposure weights of the one or more mutational signatures are calculated using a signature matrix comprising one or more mutational signatures.

5. The method of claim 1, wherein the presence of cancer in the patient is detected from the one or more exposure weights of the one or more mutational signatures using a semi-supervised approach, wherein the one or more exposure weights of the one or more mutational signatures are calculated using a signature matrix comprising one or more mutational signatures.

6. The method of claim 1, wherein the presence of the cancer in the patient is detected from the one or more exposure weights of the one or more mutational signatures using an unsupervised approach, wherein the one or more exposure weights of the one or more mutational signatures and a signature matrix are jointly calculated.

7. The method of claim 1, wherein the presence of cancer in the patient is detected when the one or more exposure weights for the one or more mutational signatures exceeds a threshold value.

8. The method of claim 1, wherein the presence of cancer in the patient is detected by performing a clustering procedure on the one or more mutational signatures.

9. The method of claim 1, wherein the presence of cancer in the patient is detected by performing a classification procedure on the one or more mutational signatures.

10. The method according to claim 1, wherein the computer is configured to generate a report that comprises the one or more exposure weights of the one or more mutational signatures.

11. The method of claim 1, wherein the computer is configured to generate a report that comprises a cancer classification.

12. The method of claim 1, wherein the computer is configured to generate a report that comprises a hierarchical clustering of signature profiles.

13. The method according to claim 1, wherein the computer comprises a communication module, and wherein the method further comprises:
transmitting the one or more mutational profiles to a remote server that is programmed to:
access a database that comprises the signature matrix;
determine the one or more exposure weights for the one or more mutational signatures; and
detect the presence of the cancer in the patient based on the one or more exposure weights of the one or more mutational signatures; and
receiving, from the remote server, a report that comprises the one or more exposure weights of the one or more mutational signatures and indicates a cancer status of the patient.

14. The method according to claim 1, wherein the computer comprises a communication module, and wherein the method further comprises:
transmitting the one or more mutational profiles to a remote server that is programmed to:
compute a signature matrix;
determine the one or more exposure weights for the one or more mutational signature; and
detect the presence of the cancer in the patient based on the one or more exposure weights of the one or more mutational signatures; and
receiving, from the remote server, a report that comprises the one or more exposure weights of the one or more mutational signatures, and indicates a cancer status of the patient.

15. A computer-implemented method for determining a cancer cell-type or tissue of origin of a cancer in a patient, the method comprising:
receiving a data set in a computer comprising a processor and a computer-readable medium, wherein the data set comprises a plurality of sequence reads obtained by sequencing a plurality of nucleic acids in a biological test sample from the patient, and wherein the computer-readable medium comprises instructions that, when executed by the processor, cause the computer to:
identify one or more somatic mutations in the biological test sample;
generate a somatic mutational profile that comprises the one or more somatic mutations;
deconvolute the somatic mutational profile into one or more mutational signatures; and
determine one or more exposure weights for one or more of the mutational signatures; and
determining the cancer cell-type or tissue of origin of the cancer in the patient based on the one or more exposure weights of the one or more mutational signatures.

16. The method of claim 15, wherein the one or more somatic mutations are identified by aligning the plurality of sequence reads to a reference genome.

17. The method of claim 15, wherein the one or more somatic mutations are identified by performing a de novo assembly procedure on a plurality of sequence reads.

18. The method of claim 15, wherein the cancer cell-type or tissue of origin of the cancer is determined from the one or more exposure weights of the one or more mutational signatures using a supervised approach, wherein the one or more exposure weights of the one or more mutational signatures is calculated using a signature matrix comprising one or more mutational signatures.

19. The method of claim 15, wherein the cancer cell-type or tissue of origin of the cancer is detected from the one or more exposure weights of the one or more mutational signatures using a semi-supervised approach, wherein the one or more exposure weights of the one or more mutational signatures are calculated using a signature matrix comprising one or more mutational signatures.

20. The method of claim 15, wherein the cancer cell-type or tissue of origin of the cancer is determined from the one or more exposure weights of the one or more mutational signatures using an unsupervised approach, wherein the one or more exposure weights of the one or more mutational signatures and a signature matrix are jointly calculated.

21. The method according to claim 15, wherein the computer is configured to generate a report that comprises the one or more exposure weights of the one or more mutational signatures.

22. The method of claim 15, wherein the computer is configured to generate a report that comprises a cancer classification.

23. The method of claim 15, wherein the computer is configured to generate a report that comprises a hierarchical clustering of signature profiles.

24. The method according to claim 15, wherein the computer comprises a communication module, and wherein the method further comprises:
transmitting the one or more mutational profiles to a remote server that is programmed to:
access a database that comprises the signature matrix; and
determine the one or more exposure weights of the one or more mutational signatures; and
receiving, from the remote server, a report that comprises the one or more exposure weights of the one or more mutational signatures and indicating the cancer cell-type or tissue of origin of the cancer in the patient.

25. The method according to claim 15, wherein the computer comprises a communication module, and wherein the method further comprises:
transmitting the one or more mutational profiles to a remote server that is programmed to:
compute a signature matrix; and
determine the one or more exposure weights for each of the one or more mutational signatures that matches a cancer-associated mutational signature in the signature matrix; and
receiving, from the remote server, a report that comprises the one or more exposure weights of the one or more mutational signatures, and indicating the tissue or origin of the cancer in the patient.

26. A computer-implemented method for determining one or more causative mutational processes of a cancer in a patient, the method comprising:
receiving a data set in a computer comprising a processor and a computer-readable medium, wherein the data set comprises a plurality of sequence reads obtained by sequencing a plurality of nucleic acids in a biological test sample from the patient, and wherein the computer-readable medium comprises instructions that, when executed by the processor, cause the computer to:
identify one or more somatic mutations in the biological test sample;
generate a somatic mutational profile that comprises the one or more somatic mutations;
deconvolute the somatic mutational profile into one or more mutational signatures; and
determine one or more exposure weights for one or more of the mutational signatures; and
determining the causative mutational process of the cancer in the patient based on the one or more exposure weights for the one or more mutational signatures.

27. The method of claim 26, wherein the one or more somatic mutations are identified by aligning the plurality of sequence reads to a reference genome.

28. The method of claim 26, wherein the one or more somatic mutations are identified by performing a de novo assembly procedure on a plurality of sequence reads.

29. The method of claim 26, wherein the one or more causative mutational processes of the cancer are determined from the one or more exposure weights of the one or more mutational signatures using a supervised approach, wherein the one or more exposure weights of the one or more mutational signatures is calculated using a signature matrix comprising one or more mutational signatures.

30. The method of claim 26, wherein the presence of cancer in the patient is detected from the one or more exposure weights of the one or more mutational signatures using a semi-supervised approach, wherein the one or more exposure weights of the one or more mutational signatures are calculated using a signature matrix comprising one or more mutational signatures.

31. The method of claim 26, wherein the one or more causative mutational processes of the cancer are determined from the one or more exposure weights of the one or more mutational signatures using an unsupervised approach, wherein the one or more exposure weights of the one or more mutational signatures and a signature matrix are jointly calculated.

32. The method according to claim 26, wherein the computer is configured to generate a report that comprises the one or more exposure weights of the one or more mutational signatures.

33. The method of claim 26, wherein the computer is configured to generate a report that comprises a cancer classification.

34. The method of claim 26, wherein the computer is configured to generate a report that comprises a hierarchical clustering of signature profiles.

35. The method according to claim 26, wherein the computer comprises a communication module, and wherein the method further comprises:
transmitting the one or more mutational profiles to a remote server that is programmed to:
access a database that comprises the signature matrix; and
determine the one or more exposure weights for the one or more mutational signatures; and
receiving, from the remote server, a report that comprises the one or more exposure weights of the one or more mutational signatures, and indicates the causative mutational process of the cancer in the patient.

36. The method according to claim 26, wherein the computer comprises a communication module, and wherein the method further comprises:
transmitting the one or more mutational profiles to a remote server that is programmed to:
compute a signature matrix; and
determine the one or more exposure weights for each of the one or more mutational signatures; and
receiving, from the remote server, a report that comprises the one or more exposure weights of the one or more mutational signatures, and indicates the causative mutational process of the cancer in the patient.

37. A method for therapeutically classifying a cancer patient into one or more of a plurality of treatment categories, the method comprising:
receiving a data set in a computer comprising a processor and a computer-readable medium, wherein the data set comprises a plurality of sequence reads obtained by sequencing a plurality of nucleic acids in a biological test sample from the patient, and wherein the computer-readable medium comprises instructions that, when executed by the processor, cause the computer to:
identify one or more somatic mutations in the biological test sample;
generate a somatic mutational profile that comprises the one or more somatic mutations;
deconvolute the somatic mutational profile into one or more mutational signatures; and
determine one or more exposure weights for one or more of the mutational signatures; and
classifying the patient into one or more of the plurality of treatment categories based on the one or more exposure weights of the one or more mutational signatures.

38. The method of claim 37, wherein the one or more somatic mutations are identified by aligning the plurality of sequence reads to a reference genome.

39. The method of claim 37, wherein the one or more somatic mutations are identified by performing a de novo assembly procedure on a plurality of sequence reads.

40. The method of claim 37, wherein the cancer patient is therapeutically classified into one or more of the plurality of treatment categories from the one or more exposure weights of the one or more mutational signatures using a supervised approach, wherein the one or more exposure weights of the one or more mutational signatures is calculated using a signature matrix comprising one or more mutational signatures.

41. The method of claim 37, wherein the presence of cancer in the patient is detected from the one or more exposure weights of the one or more mutational signatures using a semi-supervised approach, wherein the one or more exposure weights of the one or more mutational signatures are calculated using a signature matrix comprising one or more mutational signatures.

42. The method of claim 37, wherein the cancer patient is therapeutically classified into one or more of the plurality of treatment categories from the one or more exposure weights of the one or more mutational signatures using an unsupervised approach, wherein the one or more exposure weights of the one or more mutational signatures and a signature matrix are jointly calculated.

43. The method according to claim 37, wherein the computer is configured to generate a report that comprises the one or more exposure weights of the one or more mutational signatures.

44. The method of claim 37, wherein the computer is configured to generate a report that comprises a cancer classification.

45. The method of claim 37, wherein the computer is configured to generate a report that comprises a hierarchical clustering of signature profiles.

46. The method according to claim 37, wherein the computer comprises a communication module, and wherein the method further comprises:
transmitting the one or more mutational profiles to a remote server that is programmed to:
access a database that comprises the signature matrix; and
determine the one or more exposure weights for the one or more mutational signatures; and
receiving, from the remote server, a report that comprises the one or more exposure weights of the one or more mutational signatures and classifies the patient into one or more of the plurality of treatment categories.

47. The method according to claim 37, wherein the computer comprises a communication module, and wherein the method further comprises:
transmitting the one or more mutational profiles to a remote server that is programmed to:
compute a signature matrix; and
determine the one or more exposure weights for each of the one or more mutational signatures; and
receiving, from the remote server, a report that comprises the one or more exposure weights of the one or more mutational signatures, and that classifies the patient into one or more of the plurality of treatment categories

48. The method according to any one of claims 1-47, wherein the signature matrix comprises one or more learned error signatures.

49. The method according to claim 48, wherein the one or more learned error signatures comprise a systematic error signature.

50. The method according to claim 59, wherein the systematic error signature is associated with a sequencing library preparation error, a PCR error, a hybridization capture error, a sequencing error, a defect introduced through chemically induced DNA damage, a defect introduced through mechanically induced DNA damage, or any combination thereof.

51. The method according to claim 58, wherein the one or more learned error signatures in the signature matrix comprise a plurality of different feature probabilities.

52. The method according to any one of claims 1-47, wherein the signature matrix comprises one or more healthy aging signatures.

53. The method according to claim 52, wherein the one or more healthy aging signatures in the signature matrix comprise a plurality of different feature probabilities.

54. The method according to any one of claims 1-47, further comprising removing one or more learned error signatures and/or one or more healthy aging signatures from the somatic mutational profile.

55. The method according to claim 1, wherein the somatic mutational profile comprises: an upstream sequence context of a base substitution mutation, a downstream sequence context of a base substitution mutation, an insertion, a deletion (Indel), a somatic copy number alteration (SCNA), a translocation, a genomic methylation status, a chromatin state, a sequencing depth of coverage, an early versus late replicating region, a sense versus antisense strand, an inter mutation distance, a variant allele frequency, a fragment start/stop, a fragment length, a gene expression status, or any combination thereof.

56. The method according to claim 1, wherein the somatic mutational profile comprises a sequence context.

57. The method according to claim 56, wherein the sequence context comprises one or more base substitution mutations, insertions, deletions, somatic copy number alterations, translocations, or any combination thereof.

58. The method according to claim 56, wherein the sequence context comprises a genomic methylation status.

59. The method according to claim 56, wherein the sequence context comprises a gene expression status.

60. The method according to claim 56, wherein the sequence context is selected from a region of a nucleic acid that ranges from about 2 to about 40 bp of base substitution mutations.

61. The method according to claim 56, wherein the sequence context comprises a triplet sequence context, a quadruplet sequence context, a quintuplet sequence context, a sextuplet sequence context, or a septuplet sequence context of base substitution mutations.

62. The method according to claim 48, wherein the sequence context comprises a triplet sequence context of base substitution mutations.

63. The method according to any one of claims 56-62, wherein the sequence context is an upstream sequence context, a downstream sequence context, or a combination thereof.

64. The method according to claim 1, wherein the one or more somatic mutations comprise a driver mutation.

65. The method according to claim 1, wherein the one or more somatic mutations comprise a passenger mutation.

66. The method according to any one of claims 1-65, wherein sequencing the plurality of nucleic acids in the biological test sample comprises conducting a next-generation sequencing procedure.

67. The method according to any one of claims 1-65, wherein sequencing the plurality of nucleic acids in the biological test sample comprises conducting a sequencing by synthesis procedure.

68. The method according to any one of claims 1-65, wherein sequencing the plurality of nucleic acids in the biological test sample comprises conducting a pyrosequencing procedure.

69. The method according to any one of claims 1-65, wherein sequencing the plurality of nucleic acids in the biological test sample comprises conducting an ion semiconductor sequencing procedure.

70. The method according to any one of claims 1-65, wherein sequencing the plurality of nucleic acids in the biological test sample comprises conducting a single-molecule real-time sequencing procedure.

71. The method according to any one of claims 1-65, wherein sequencing the plurality of nucleic acids in the biological test sample comprises conducting a sequencing by ligation procedure.

72. The method according to any one of claims 1-65, wherein sequencing the plurality of nucleic acids in the biological test sample comprises conducting a nanopore sequencing procedure.

73. The method according to any one of claims 1-65, wherein sequencing the plurality of nucleic acids in the biological test sample comprises conducting a massively parallel sequencing procedure.

74. The method according to claim 73, wherein the massively parallel sequencing procedure comprises a sequencing by synthesis procedure that employs one or more reversible dye terminators.

75. The method according to any one of claims 1-65, wherein sequencing the plurality of nucleic acids in the biological test sample comprises conducting a sequencing by ligation procedure.

76. The method according to any one of claims 1-65, wherein sequencing the plurality of nucleic acids in the biological test sample comprises conducting a single molecule sequencing procedure.

77. The method according to any one of claims 1-65, wherein sequencing the plurality of nucleic acids in the biological test sample comprises conducting a paired end sequencing procedure.

78. The method according to any one of claims 1-77, further comprising performing an amplification procedure prior to sequencing the plurality of nucleic acids in the biological test sample.

79. The method according to any one of the preceding claims, wherein the nucleic acids in the biological test sample comprise DNA.

80. The method according to any one of the preceding claims, wherein the nucleic acids in the biological test sample comprise RNA.

81. The method according to any one of the preceding claims, wherein the nucleic acids in the biological test sample comprise cell-free DNA (cfDNA).

82. The method according to any one of the preceding claims, wherein the nucleic acids in the biological test sample comprise circulating tumor DNA (ctDNA).

83. The method according to any one of the preceding claims, wherein the nucleic acids in the biological test sample comprise nucleic acids from cancerous and non-cancerous cells.

84. The method according to any one of the preceding claims, wherein the biological test sample comprises a biological fluid.

85. The method according to claim 84, wherein the biological fluid comprises blood.

86. The method according to claim 84, wherein the biological fluid comprises plasma.

87. The method according to claim 84, wherein the biological fluid comprises serum.

88. The method according to claim 84, wherein the biological fluid comprises urine.

89. The method according to claim 84, wherein the biological fluid comprises saliva.

90. The method according to claim 84, wherein the biological fluid comprises pleural fluid.

91. The method according to claim 84, wherein the biological fluid comprises pericardial fluid.

92. The method according to claim 84, wherein the biological fluid comprises cerebrospinal fluid (CSF).

93. The method according to claim 84, wherein the biological fluid comprises peritoneal fluid.

94. The method according to any one of claims 1-83, wherein the biological test sample comprises a tissue biopsy.

95. The method according to claim 94, wherein the tissue biopsy is a cancerous tissue biopsy.

96. The method according to claim 94, wherein the tissue biopsy is a healthy tissue biopsy.

97. The method according to any one of the preceding claims, wherein the cancer comprises a carcinoma, a sarcoma, a myeloma, a leukemia, a lymphoma, a blastoma, a germ cell tumor, or any combination thereof.

98. The method according to claim 97, wherein the carcinoma is an adenocarcinoma.

99. The method according to claim 97, wherein the carcinoma is a squamous cell carcinoma.

100. The method according to claim 97, wherein the carcinoma is selected from the group consisting of: small cell lung cancer, non-small-cell lung, nasopharyngeal, colorectal, anal, liver, urinary bladder, testicular, cervical, ovarian, gastric, esophageal, head-and-neck, pancreatic, prostate, renal, thyroid, melanoma, and breast carcinoma.

101. The method according to claim 97, wherein the breast cancer is hormone receptor negative breast cancer or triple negative breast cancer.

102. The method according to claim 97, wherein the sarcoma is selected from the group consisting of: osteosarcoma, chondrasarcoma, leiomyosarcoma, rhabdomyosarcoma, mesothelial sarcoma (mesothelioma), fibrosarcoma, angiosarcoma, liposarcoma, glioma, and astrocytoma.

103. The method according to claim 97, wherein the leukemia is selected from the group consisting of: myelogenous, granulocytic, lymphatic, lymphocytic, and lymphoblastic leukemia.

104. The method according to claim 97, wherein the lymphoma is selected from the group consisting of: Hodgkin's lymphoma and Non-Hodgkin's lymphoma.

105. A computer-implemented method for constructing a signature matrix of cancer-associated mutational signatures for a plurality of different cancer types, the method comprising:
(a) compiling a plurality of sequence reads obtained from a plurality of cancer patients with a known cancer status across a plurality of different cancer types to generate an observed matrix of mutational profiles;
(b) deconvoluting the observed matrix into a plurality of cancer-associated mutational signatures;
(c) identifying one or more exposure weights for each of the cancer-associated mutational signatures;
(d) assigning a cancer type to each of the cancer-associated mutational signatures; and
(e) assembling the plurality of cancer-associated mutational signatures into a matrix to construct the signature matrix.

106. A computer-implemented method for constructing a learned error signature matrix, the method comprising:
(a) compiling a plurality of sequence reads obtained from a plurality of samples with known errors to generate an observed matrix;
(b) deconvoluting the observed matrix into a plurality of error signatures;
(c) identifying one or more exposure weights for each of the error signatures;
(d) assigning an error signature type to each of the error signatures; and
(e) assembling the error signatures into a matrix to construct the learned error signature matrix.

107. The method according to claim 106, wherein the learned error signature matrix comprises a systematic error signature.

108. The method according to claim 107, wherein the systematic error signature is associated with a sequencing library preparation error, a nucleic acid defect, a PCR error, a hybridization capture error, a sequencing error, or any combination thereof.

109. A computer-implemented method for constructing a healthy aging signature matrix, the method comprising:
(a) compiling a plurality of sequence reads obtained from a plurality of patients with a known healthy aging status to generate an observed matrix of mutational profiles;
(b) deconvoluting the observed matrix into one or more healthy aging signatures;
(c) identifying one or more exposure weights for the one or more healthy aging signatures;
(d) assigning a healthy aging signature type to the one or more healthy aging signatures; and
(e) assembling the healthy aging signatures into a matrix to construct the healthy aging signature matrix.

110. The method according to any one of claims 105-109, wherein decomposing the matrix comprises applying a machine learning approach.

111. The method according to claim 110, wherein the machine learning approach comprises a non-negative matrix factorization (NMF) procedure.

112. The method according to claim 110, wherein the machine learning approach comprises a principal components analysis (PCA) procedure.

113. The method according to claim 110, wherein the machine learning approach comprises a vector quantization (VQ) procedure.

114. The method according to any one of claims 105-109, wherein one or more of the cancer-associated mutational signatures comprises a sequence context.

115. The method according to claim 114, wherein the sequence context comprises one or more base substitution mutations, insertions, deletions, somatic copy number alterations, translocations, or any combination thereof.

116. The method according to claim 114, wherein the sequence context comprises a genomic methylation status.

117. The method according to claim 114, wherein the sequence context comprises a gene expression status.

118. The method according to claim 114, wherein the sequence context comprises a triplet sequence context of base substitution mutations.

119. The method according to any one of claims 114-118, wherein the sequence context is an upstream sequence context, a downstream sequence context, or a combination thereof.

120. The method according to claim 105, wherein one or more of the cancer-associated mutational signatures comprises a driver mutation.

121. The method according to claim 105, wherein one or more of the cancer-associated mutational signatures comprises a passenger mutation.

122. A computer-implemented method for detecting the presence of a cancer in a patient, the method comprising:
compiling a plurality of sequence reads obtained from a plurality of cancer patients with a known cancer status across a plurality of different cancer types to generate an observed matrix in a computer comprising a processor and a computer-readable medium;
deconvoluting the observed matrix into one or more cancer-associated mutational signatures;
identifying one or more exposure weights for the one or more cancer-associated mutational signatures;
assembling the cancer-associated mutational signatures into a matrix to construct the signature matrix;
receiving a data set in the computer, wherein the data set comprises a plurality of sequence reads obtained by sequencing a plurality of nucleic acids in a biological test sample from the patient, and wherein the computer-readable medium comprises instructions that, when executed by the processor, cause the computer to:
identify one or more somatic mutations in the biological test sample;
generate a somatic mutational profile that comprises the one or more somatic mutations;
deconvolute the somatic mutational profile into one or more mutational signatures; and
determine one or more exposure weights for one or more of the mutational signatures; and
detecting the presence of the cancer in the patient based on the one or more exposure weight of the one or more mutational signatures.

123. A computer-implemented method for determining a cancer cell-type or tissue of origin of a cancer in a patient, the method comprising:
compiling a plurality of sequence reads obtained from a plurality of cancer patients with a known cancer status across a plurality of different cancer types to generate an observed matrix in a computer comprising a processor and a computer-readable medium;
deconvoluting the observed matrix into one or more cell-type or tissue-associated mutational signatures;
identifying one or more exposure weights for the one or more cell-type or tissue-associated mutational signatures;
assigning a cancer cell-type or tissue of origin designation to the one or more cell-type or tissue-associated mutational signatures;
assembling the one or more cell-type or tissue-associated mutational signatures into a matrix to construct the signature matrix;
receiving a data set in the computer, wherein the data set comprises a plurality of sequence reads obtained by sequencing a plurality of nucleic acids in a biological test sample from the patient, and wherein the computer-readable medium comprises instructions that, when executed by the processor, cause the computer to:
identify one or more somatic mutations in the biological test sample;
generate a somatic mutational profile that comprises the one or more somatic mutations;
deconvolute the somatic mutational profile into one or more mutational signatures; and
determine one or more exposure weights for the one or more mutational signatures; and
determining the cell-type or tissue of origin of the cancer in the patient based on the one or more exposure weights of the one or more mutational signatures.

124. A computer-implemented method for therapeutically classifying a cancer patient into one or more of a plurality of treatment categories, the method comprising:
compiling a plurality of sequence reads obtained from a plurality of cancer patients with a known cancer status across a plurality of different cancer types to generate an observed matrix in a computer comprising a processor and a computer-readable medium;
deconvoluting the observed matrix into one or more cancer-associated mutational signatures;
identifying one or more exposure weights for the one or more cancer-associated mutational signatures;
assigning a cancer type and a treatment category to the one or more cancer-associated mutational signatures;
assembling the cancer-associated mutational signatures into a matrix to construct the signature matrix;
receiving a data set in the computer, wherein the data set comprises a plurality of sequence reads obtained by sequencing a plurality of nucleic acids in a biological test sample from the patient, and wherein the computer-readable medium comprises instructions that, when executed by the processor, cause the computer to:
identify one or more somatic mutations in the biological test sample;
generate a somatic mutational profile that comprises the one or more somatic mutations;
deconvolute the somatic mutational profile into one or more mutational signatures; and
determine one or more exposure weights for the one or more mutational signatures; and
classifying the patient into one or more of the treatment categories based on the one or more exposure weights of the one or more mutational signatures.

125. The method according to any one of claims 122-124, wherein the one or more somatic mutations are identified by aligning the plurality of sequence reads to a reference genome.

126. The method according to any one of claims 122-124, wherein the one or more somatic mutations are identified by performing a de novo assembly procedure on a plurality of sequence reads.

127. The method according to any one of claims 105-126, wherein the sequence reads are obtained from nucleic acids in the biological test sample, and wherein the nucleic acids comprise DNA.

128. The method according to any one of claims 105-126, wherein the sequence reads are obtained from nucleic acids in the biological test sample, and wherein the nucleic acids comprise RNA.

130. The method according to any one of claims 105-126, wherein the sequence reads are obtained from nucleic acids in the biological test sample, and wherein the nucleic acids comprise cell-free DNA (cfDNA).

131. The method according to any one of claims 105-126, wherein the sequence reads are obtained from nucleic acids in the biological test sample, and wherein the nucleic acids comprise circulating tumor DNA (ctDNA).

132. The method according to any one of claims 105-126, wherein the sequence reads are obtained from nucleic acids in the biological test sample, and wherein the nucleic acids comprise nucleic acids from cancerous and non-cancerous cells.


SEQUENCE LISTING (7 sequences)

SEQ ID NO: 1 - DNA, 10 bp, Artificial Sequence
  Description of Artificial Sequence Synthetic oligonucleotide
accccacccc

SEQ ID NO: 2 - DNA, 25 bp, Artificial Sequence
  Description of Artificial Sequence Synthetic oligonucleotide
ctcccttcctcactgggattcagag

SEQ ID NO: 3 - DNA, 10 bp, Artificial Sequence
  Description of Artificial Sequence Synthetic oligonucleotide
cttttttttt

SEQ ID NO: 4 - DNA, 12 bp, Artificial Sequence
  Description of Artificial Sequence Synthetic oligonucleotide
cttttttttttt

SEQ ID NO: 5 - DNA, 14 bp, Artificial Sequence
  Description of Artificial Sequence Synthetic oligonucleotide
cttttttttttttt

SEQ ID NO: 6 - DNA, 15 bp, Artificial Sequence
  Description of Artificial Sequence Synthetic oligonucleotide
ctttttttttttttt

SEQ ID NO: 7 - DNA, 10 bp, Artificial Sequence
  Description of Artificial Sequence Synthetic oligonucleotide
gttttttttt